(19) United States
(12) Patent Application Publication
(10) Pub. No.: US 2014/0039279 A1
(43) Pub. Date: Feb. 6, 2014

(54) ACOUSTIC PALPATION USING NON-INVASIVE ULTRASOUND TECHNIQUES FOR IDENTIFICATION OF TARGET SITES AND ASSESSMENT OF CHRONIC PAIN DISORDERS

(71) Applicants: UNIVERSITY OF WASHINGTON, Seattle, WA (US); Physiosonics, Inc., Bellevue, WA (US)

(72) Inventors: Jeffrey G. Jarvik, Seattle, WA (US); Pierre Mourad, Seattle, WA (US); Michel Kliot, San Francisco, CA (US); Robert C.A. Frederickson, Vancouver (CA); Abbi M McClintic, Monroe, WA (US); Trevor C. Dickey, Durham, NC (US); Michael Gofeld, Seattle, WA (US)

(73) Assignees: UNIVERSITY OF WASHINGTON, Seattle, WA (US); PHYSIOSONICS, INC., Bellevue, WA (US)

(21) Appl. No.: 14/051,205

(22) Filed: Oct. 10, 2013

Related U.S. Application Data
(63) Continuation of application No. 13/161,214, filed on Jun. 15, 2011.
Application No. 13/161,214 is a continuation-in-part of application No. 12/563,060, filed on Sep. 18, 2009, now abandoned.
Application No. 12/563,060 is a continuation-in-part of application No. 11/050,993, filed on Feb. 4, 2005.
Application No. 11/050,993 is a continuation of application No. 09/995,897, filed on Nov. 28, 2001, now Pat. No. 6,875,176.
(60) Provisional application No. 61/355,034, filed on Jun. 15, 2010.
(60) Provisional application No. 61/192,650, filed on Sep. 19, 2008.
(60) Provisional application No. 60/253,959, filed on Nov. 28, 2000.

(51) Int. Cl. A61B 5/00; A61B 8/00

(52) U.S. Cl. CPC A61B 5/4827; A61B 8/00
USPC 600/301; 600/552; 600/438; 604/506; 604/512

(57) ABSTRACT

Methods and systems for identifying and spatially localizing tissues having certain physiological properties or producing certain biological responses, such as the sensation of pain, in response to the application of intense focused ultrasound (acoustic probing or palpation) are provided. In some embodiments, targeted acoustic probing is employed to identify the scope and severity of chronically painful sensitized tissue areas, and of chronic pain disorders. In other applications, targeted acoustic probing is used to localize nerves and other sensitized tissues for guidance of needles and other delivery devices, and for delivery of anesthetic, analgesic or therapeutic compositions.

### REFERENCE TO PRIORITY APPLICATIONS

This application is a continuation of U.S. patent application Ser. No. 13/161,214, filed Jun. 15, 2011, which claims priority to U.S. Provisional Patent Application No. 61/355,034 filed Jun. 15, 2010 and which is a continuation-in-part of U.S. patent application Ser. No. 12/563,060, filed Sep. 18, 2009, which claims priority to U.S. Provisional Patent Application No. 61/192,650, filed Sep. 19, 2008. This application is also a continuation-in-part of U.S. patent application Ser. No. 11/050,993, filed Feb. 4, 2005, which is a continuation of U.S. patent application Ser. No. 09/995,897, filed Nov. 28, 2001 and issued as U.S. Pat. No. 6,875,176 on Apr. 4, 2005, which claims priority to U.S. Provisional Patent Application No. 60/253,959, filed Nov. 28, 2000. The disclosures of these priority applications are incorporated by reference herein in their entireties.

### STATEMENT REGARDING FEDERALLY SPONSORED RESEARCH

Subject matter disclosed in this application was supported by federally sponsored research and development funding. The U.S. Government may have certain rights in the invention as provided for by the terms of NIH Grant 1R41NS049719-01 and VA Merit Rehab Grant #F3624-R.

### TECHNICAL FIELD OF THE INVENTION

In one aspect, the present invention relates to methods and systems for identifying and spatially localizing tissues having certain physiological properties or producing certain biological responses, such as the sensation of pain, in response to the application of focused ultrasound (acoustic probing or palpation), and for assessing chronic pain disorders. In some embodiments, targeted acoustic probing may be guided or visualized using imaging techniques such as ultrasound imaging or other types of non-invasive imaging techniques. Treatment of identified target sites may be provided using therapeutic ultrasound techniques. Methods and systems of the present invention may also incorporate one or more diagnostic and/or therapeutic ultrasound techniques with one or more other diagnostic and/or therapeutic modalities and may employ diagnostic or diagnostic-type ultrasound probes for targeted acoustic probing.

### BACKGROUND OF THE INVENTION

In the field of medical imaging, ultrasound may be used in various modes to produce images of objects or structures within a patient. In a transmission mode, an ultrasound transmitter is placed on one side of an object (e.g. a body portion) and ultrasound beams are transmitted into the object (body). Ultrasound receive beams are acquired by an ultrasound receiver. An image may be produced in which the brightness of each image pixel is a function of the amplitude of the ultrasound that reaches the receiver (attenuation mode), or the brightness of each pixel may be a function of the time required for the sound to reach the receiver (time-of-flight mode). Alternatively, if the receiver is positioned on the same side of the object as the transmitter, an image may be produced in which the pixel brightness is a function of the amplitude of reflected ultrasound (reflection or backscatter or echo mode). In a Doppler mode of operation, the tissue (or object) is imaged by measuring the phase shift of the ultrasound reflected from the tissue (or object) back to the receiver.

When used for imaging, ultrasound transducers are provided with several piezoelectric elements arranged in an array and driven by different voltages. By controlling the phase and amplitude of the applied voltages, ultrasound waves combine to produce a net ultrasound wave that travels along a desired beam direction and is focused at a selected point along the beam. By controlling the phase and the amplitude of the applied voltages, the focal point of beams can be moved in a plane to scan the subject. Ultrasound imaging systems and transducers are well known in the art.

An acoustic radiation force is exerted by an acoustic wave on an object in its path. The use of acoustic radiation forces produced by an ultrasound transducer has been proposed in connection with tissue hardness measurements. See Sugimoto et al., “Tissue Hardness Measure Using the Radiation Force of Focused Ultrasound”, IEEE Ultrasonics Symposium, pp. 1377-80, 1990. This publication describes an experiment in which a pulse of focused ultrasonic radiation is applied to deform the object at the focal point of the transducer. The deformation is measured using a separate pulse-echo ultrasonic system. Measurements of tissue hardness are made based on the amount or rate of object deformation as the acoustic force is continuously applied, or by the rate of relaxation of the deformation after the force is removed.

Another system is disclosed by T. Sato, et al., “Imaging of Acoustical Nonlinear Parameters and Its Medical and Industrial Applications: A Viewpoint as Generalized Percussion,” Acoustical Imaging, Vol. 20, pg. 9-18, Plenum Press, 1993. In this system, a lower frequency wave (350 kHz) is used as a percussion force, and an ultrasonic wave (5 MHz) is used in a pulse-echo mode to produce an image of the subject. The percussion force perturbs second order nonlinear interactions in tissues, which may reveal more structural information than conventional ultrasound pulse-echo systems.

Fatemi and Greenleaf reported an imaging technique that uses acoustic emission to map the mechanical response of an object to local cyclic radiation forces produced by interfering ultrasound beams. The object is probed by arranging the intersection of two focused, continuous-wave ultrasound beams of different frequencies at a selected point on the object. Interference in the intersection region of the two beams produces modulation of the ultrasound energy density, which creates a vibration in the object at the selected region. The vibration produces an acoustic field that can be measured. The authors speculate that ultrasound-stimulated vibro-acoustic spectrography has potential applications in the non-destructive evaluation of materials, and for medical imaging and noninvasive detection of hard tissue inclusions, such as the imaging of arteries with calcification, detection of breast microcalcifications, visualization of hard tumors, and detection of foreign objects.

U.S. Pat. Nos. 5,903,516 and 5,921,928 (Greenleaf et al.) disclose a method and system for producing an acoustic radiation force at a target location by directing multiple high frequency sound beams to intersect at the desired location. A variable amplitude radiation force may be produced using variable, high frequency sound beams, or by amplitude modulating a high frequency sound beam at a lower, baseband frequency. The mechanical properties of an object, or the presence of an object, may be detected by analyzing the acoustic wave that is generated from the object by the applied acoustic radiation force. An image of the object may be produced by scanning the object with high frequency sound beams and analyzing the acoustic waves generated at each scanned location. The mechanical characteristics of an object may also be assessed by detecting the motion produced at the intersections of high frequency sound beams and analyzing the motion using Doppler ultrasound and nuclear magnetic resonance imaging techniques. Variations in the characteristics of fluids (e.g. blood), such as fluid temperature, density and chemical composition can also be detected by assessing changes in the amplitude of the beat frequency signal. Various applications are cited, including detection of atherosclerosis, detection of gas bubbles in fluids, measurement of contrast agent concentration in the blood stream, object position measurement, object motion and velocity measurement, and the like. An imaging system is also disclosed.

The use of ultrasound in therapeutic regimen for heating tissues is well established. In general, ultrasound devices for the treatment and rehabilitation of muscle injuries and other soft tissue damage use low intensity, long duration and weakly focused ultrasound. Ultrasound heads having various dimensions, shapes and using various ultrasound frequencies and other operational parameters are known. Low intensity acoustic shock waves are administered in shock wave therapy (SWT) for treatment of arthritis, plantar faciitis and bone abnormalities. The use of high intensity focused ultrasound (HIFU) in medicine and physiology for the local destruction or cauterization of deep-seated tissues is also well known. High acoustic intensity shock waves are used, for example, in lithotripsy.

The application of focused ultrasound may thus induce changes or biological responses remotely in structures and tissues and has been reported to induce pain. Davies et al. showed that short pulses of focused ultrasound stimulated the superficial and deep-seated receptor structures of human tissues and induced different somatosensory sensations including, in particular, pain sensations. Threshold values of ultrasound parameters corresponding to the induction of pain for different frequencies, stimulus duration and localizations of the different types of tissue are also given. Davies et al., Application of focused ultrasound for research on pain, Pain, 67:17-27 (1996)-1996 International Association for the Study of Pain. Wright et al. have also shown that application of focused ultrasound elicits temporal summation of pain in skin, joint and muscle tissue. A. Wright et al., Temporal summation of pain from skin, muscle and joint following nociceptive ultrasonic stimulation in humans, Exp Brain Res 144:475-482 (2002).

Pain is a frequent presenting symptom of numerous medical conditions and is often the first sign that something is wrong with a patient. However, in up to 85% of cases, neither a physical examination nor any diagnostic tests are helpful in pinpointing the anatomical structures responsible for generating the pain (Jarvik and Deyo, 2002). Physical examination of a patient can be used in diagnosing a host of medical maladies, including a number of pain syndromes. Unfortunately, physical examination maneuvers, including tissue palpation, is generally unsuccessful in localizing the source of pain from deep tissues and nonspecific for attributing pathologies to particular anatomic structures due, in part, to an inability to stimulate small deep structures selectively. Existing diagnostic modalities, such as X-rays, computed tomography (CT), and magnetic resonance imaging (MRI) studies are exquisitely sensitive in identifying and localizing subtle anatomic abnormalities and are often used to examine patients presenting with pain having an unidentified source or cause. A patient's pain symptoms often correlate poorly with anatomic abnormalities or anomalies identified by the various diagnostic imaging techniques, however, and deep tissue and joint pain remains difficult to localize and accurately diagnose.

There are many common conditions that would benefit from techniques for increasing the specificity and localization of pain. Low back pain (LBP) is a prime example of one common condition. The lifetime incidence of LBP is reported to be 60-90%, with an annual incidence of 5%. Each year, 14% of new patient visits to primary care physicians are for LBP, and nearly 13 million physician visits are related to complaints of chronic LBP, according to the National Center for Health Statistics. Unfortunately, it is difficult to identify the exact source of pain: several constituent components of a complex structure may be intimately adjoining, yet only one may be the source. While half of the American work force reports back pain, only about 20% of those cases result in a specific diagnosis of the source of pain. X-rays, computed tomography (CT) and magnetic resonance imaging (MRI) are the major diagnostic imaging tests for patients with low back pain and, while they can exquisitely depict anatomic abnormalities, the correlations between anatomic findings and patient symptoms are moderate at best.

In recent years, back pain specialists have begun to rely on invasive provocative tests in attempts to identify the “pain generator.” Physicians insert needles into discs for discography to provoke pain and into facet and sacroiliac joints to provoke and then relieve pain through the injection of local anesthetics and steroids. These tests are frequently uncomfortable for the patient and carry the risk of infection, bleeding and contrast reaction. As with magnetic resonance and CT scanning the specificity of these tests has been questioned. And, therapy may be radically different depending on the results of these tests.

Osteoporotic compression fractures are highly prevalent in the elderly. The incidence is 700,000 fractures per year, generating 160,000 physician visits annually and over 5 million restricted activity days. Until recently, there were no good options for treatment. Vertebroplasty, which is the percutaneous injection of methylmethacrylate into the vertebral body is a new, promising treatment for these fractures. But in patients with multiple fractures, identifying and localizing the painful fracture is often difficult. Palpation on physical examination, bone scans and MRI have all been used, with varying degrees of success, in attempts to localize the painful fracture(s).

Identifying and localizing the source(s) of pain emanating from internal structures and organs is also important. Localization of source(s) of pain in the abdominal cavity is notoriously difficult. The diagnosis of appendicitis, for example, is difficult and imprecise because it's difficult to palpate deep internal tissues, such as the appendix. Despite the use of advanced diagnostic imaging techniques such as CT and ultrasound, a recent review in JAMA demonstrated no change in the false positive rate demonstrated at appendectomy. Manual probing or palpation of the abdomen, with its poor specificity and patient discomfort, is still a standard test, with mixed results. Identifying and localizing the source of pain emanating from inflamed or diseased internal tissues and organs is similarly difficult and unreliable.

In the conditions described above, pain symptoms signal a problem but frequently do not pinpoint the location of that problem. In the case of back and joint pain, and in the case of pain caused by inflammation, infection or disease, such as appendicitis, cholecystitis, pancreatitis, pelvic inflammatory disease, and other conditions, there is a need to precisely, reliably and in a non-invasive manner stimulate individual constituent pieces or areas of a complex structure within the body (e.g. discs, vertebral body, lamina and facets of the spine) to identify and spatially locate the source(s) of the pain. U.S. Pat. No. 6,875,176, PCT Publication PCT/US01/044433, and U.S. Patent Publication 2006/0079773 disclose methods for localizing a physiological condition or biological response by administering ultrasound pulses to a plurality of targeted tissue sites in a subject using a focused acoustic probing technique, and acquiring data relating to a physiological condition or biological response (such as the subjective sensation of pain) induced by the ultrasound pulse(s). The present invention is directed to methods and systems for localizing physiological conditions and/or biological responses, such as pain, with sensitivity and specificity.

### SUMMARY OF THE INVENTION

Application of intense focused ultrasound (iFU) to a physiological structure or tissue produces acoustic radiation force(s) which may produce transient displacement of tissue and/or temperature change(s) and/or cavitation at the focus in the tissue. The acoustic pressure front is focused internally of an application surface. When a single element, curved ultrasound transducer is used, the ultrasound focus lies very near the geometric focus of the ultrasound source. In multiple element ultrasound transducer arrays, the ultrasound focus may be fixed or adjustable using beam steering techniques, for example, that are well known in the art.

Methods and systems of the present invention apply intense focused ultrasound (iFU) pulses to assess, localize and monitor various clinical parameters, and to diagnose, localize and monitor various conditions, responses and disease states. The methods and systems are useful, for example, for non-invasively (acoustically) probing targeted tissue sites to pinpoint the spatial location of localized tissue producing biological conditions and eliciting biological responses, such as pain, that may be associated with damaged or inflamed tissue or an underlying disease process. Application of focal acoustic (ultrasound) palpations of an appropriate magnitude, frequency, intensity, duration and/or pulse repetition rate to a target site that includes inflamed or damaged tissue, for example, evokes the sensation of pain in a subject, while application of the same intense focused ultrasound palpations to tissue sites that are not damaged does not produce the sensation of pain, or produces a qualitatively different sensation. Experimental work also indicates that application of intense focused ultrasound pulses is useful for identifying and spatially locating target tissue sites producing peripheral neuropathic pain, or neurologically induced pain, and methods and systems of the present invention are therefore also directed to the identification and localization of target sites that produce neurologically induced pain.

Painful sites within larger sites of undifferentiated pain may be identified and localized using targeted application of focal acoustic pulses (e.g., intense focused ultrasound). The source of joint pain may be identified and localized, for example, to a particular tissue (e.g. muscle, bone, nerve, connective tissue, etc.) or a particular tissue site. In many circumstances, a tissue site may not be terribly painful, but it may be enlarged or otherwise abnormal. Enlarged tissue sites may result, for example, from tumors, other abnormal growths, inflamed tissue, or the like. Cancerous nodes are generally not painful, while enlarged nodes secondary to inflammatory conditions generally are painful. Thus, acoustic probing using the techniques described herein may provide a differential diagnosis, for example, of benign versus metastatic lymphadenopathy in patients with known head and neck primary tumors. This technique is also useful for providing a differential diagnosis in other anatomic locations, such as the mediastinum and the pelvis.

A single acoustic stimulation, when applied repeatedly within frequency band of 1-20 Hz can produce an increasing number of action potentials within the spinal cord, referred to as “windup.” Such a repeated stimulus can also produce more intense sensation, described as “temporal summation.” Administration of targeted acoustic palpations (e.g., iFU) using methods and systems of the present invention provides a non-invasive methodology for inducing wind-up and temporal summation when applied in deep tissue. The use of targeted acoustic palpations to stimulate deep tissue sites has the advantage of eliminating, or significantly reducing, concomitant stimulation of skin and shallow subcutaneous tissue, which may present confounding factors when stimulation protocols employ electrical, thermal or mechanical stimulation, for example, which are generally far less targeted in their stimulatory effects.

Methods and systems of the present invention may thus also be used in the assessment and treatment of patients with chronic pain syndromes, such as fibromyalgia and other myofascial pain syndromes. Application of iFU can induce temporal summation at tissue sites, and can be used to identify both the presence of central sensitization and peripheral sources when sub-clinical stimulation is applied to the sensitized receptive field. Application of focal acoustic (ultrasound) palpations of an appropriate magnitude, frequency, intensity, duration and/or pulse repetition rate to target site(s) in patients suffering from chronic pain may be used to localize and/or map the site(s) of pain generation, providing an assessment of the location and scope of a sensitized receptive field. Application of focal acoustic (ultrasound) palpations of an appropriate magnitude, frequency, intensity, duration and/or pulse repetition rate to target site(s) in patients suffering from chronic pain may provide an evidence-based conclusion and, perhaps, a definitive diagnosis of a chronic pain disorder.

Responses to the application of focal ultrasound at sensitive tissue sites in individuals suffering from chronic pain disorders may provide an assessment of the location and scope of sensitized tissue sites, and may also be used to quantify the severity, or level of pain associated with sensitized tissue sites when compared to “normal” responses or “normal” standards.

In some situations, methods and systems of the present invention may be used to assess tissue condition or pathology based on the lack of a pain response. Probing tissue that is damaged or pathological, or that has compromised nerve function, may not produce a pain response at otherwise normal threshold levels. This lack of a biological response may indicate the presence of pathological or compromised tissue. Other, non-pain sensations may also be elicited and may provide valuable information—e.g. burning, tingling, etc.

In some embodiments, biological responses elicited by acoustic probing of tissues may be detected and monitored to indicate responses of targeted tissue sites to acoustic stimulation. Biological responses that may be detected and monitored as acoustic probing is administered according to methods and systems of the present invention include, for example, respiration, heart rate, overall body temperature and tissue temperature at the target site, electrical heart activity (electrocardiogram—ECG), blood flow velocity, blood pressure, intracranial pressure (“ICP”), blood flow-related irregularities, electrical brain activity (electroencephalogram—EEG), skin conductance or impedance, and blood oxygen composition or partial pressure (O₂, CO₂). Non-invasive pressure sensing devices such as electro-optical sensors, strain gauges and pressure transducers, for example, may be used to acquire data relating to respiration and heart rate, and conventional ECG techniques and electrodes may be used to acquire data relating to heart rate, blood oxygen composition, and electrical heart activity. Pulse oximetry techniques using, for example, electro-optical sensors, may be used to acquire data relating to heart rate and blood gas composition. Standard non-invasive blood pressure detection techniques using pressure cuffs or pressure transducers may be used to acquire data relating to blood pressure. EEG electrodes and data acquisition techniques are preferably used to acquire data relating to brain activity. Non-invasive ultrasound techniques or other non-invasive modalities are preferably used to acquire data relating to blood flow properties, blood velocity, ICP, blood flow anomalies and the like, and may also be used to acquire data relating to blood pressure.

Methods and systems of the present invention are also useful for evaluating and monitoring the healing process, as well as evaluating and monitoring responses to therapeutic agents and/or protocols by monitoring the patient's biological responses (e.g., pain elicited by acoustic probing) at both generalized and targeted locations over time. In another aspect, methods and systems of the present invention are useful for evaluating a subject's level of pain sensitivity and pain thresholds, for predicting a subject's prognosis following an intervention, and for recommending treatments. There is a growing understanding that a subject's pain and sensation thresholds, in both normal and tender tissues, are generally predictive of the susceptibility of patients to develop chronic pain following an injury or intervention, as well as to predict what medical regimens (specific drugs and other interventions such as acupuncture, injections, hypnosis, etc.) may be most effective in treating that individual's acute and chronic pain.

Targeted probing of internal body structures and tissues by application of intense focused ultrasound pulses can be applied to produce acoustic palpations having a range of target areas (or volumes) and a range of different intensities remote from the tissue surface and the ultrasound transducer. For some applications, targeted application of generally focused ultrasound pulses is preferred, with acoustic palpation or probing occurring at multiple target sites sequentially (or concurrently using multiple probes or transducers) to identify and locate target sites eliciting biological responses. For other applications, it may be desirable to initially apply a larger field of view acoustic pulse (or pulse train) to probe a larger target area, and then narrow the field of view to identify and locate target sites eliciting biological responses within the larger field of view initially probed.

For assessment and/or localization of pain, etc., one or more acoustic transducer(s) may be placed in contact with or in proximity to a subject's skin overlying or in proximity to the internal site desired to be probed. Acoustic coupling of the ultrasound probe to the surface of the subject may be provided using acoustic gels, liquids, and the like, as is known in the art. The ultrasound probe may incorporate an ultrasound transducer or a plurality of transducers or one or more transducer array(s) capable of emitting ultrasound pulses and directing one or multiple ultrasound pulses to a predetermined or selectable focal point. The ultrasound source/probe provided for acoustic palpation may provide a “point source” of ultrasound, or it may provide multiple ultrasound beams that converge at a focal point or to a focal area. The focal point or area may be fixed or adjustable, and the adjustment may be performed using electrical or electronic systems by changing the orientation or position of the ultrasound sources. Adjustment of the focal point may additionally or alternatively be performed mechanically by changing the configuration of the ultrasound probe or the arrangement of transducers or transducer elements within the probe.

In another embodiment, multiple intense focused ultrasound beams may be provided from a collocated source, or from multiple sources. In this embodiment, multiple independent or independently controllable sources of intense focused ultrasound may be used to palpate tissue at a desired target site by converging the multiple beams at selected target site(s). This may improve the accuracy of targeted probing and allow delivery of higher energy intense focused ultrasound palpations to target sites without affecting surrounding tissue. In one embodiment, multiple ultrasound transducers, each capable of delivering acoustic doses sufficient to administer intense focused ultrasound pulse palpations, provide a desired intense focused ultrasound acoustic dose by combined coincident focus at a target site. Multiple ultrasound transducers (or arrays) having multiple or independently adjustable focal points may be housed in a single, integrated probe housing, or multiple transducers (or arrays) having multiple or independently adjustable focal points may be provided in multiple separate probes. Multiple probes may be adapted for manual (clinician) placement and holding, or multiple probes may be mounted on moveable mechanical structures, such as arms, that may be manipulated to position the one or more probes at desired body surfaces for probing selected target sites. The probes may be adjustable in three dimensions to facilitate placement on different body sites, and automated spatial adjustment and positioning of the probes to interrogate programmed or programmable or selectable body locations may be provided under the control of a system controller.

In another embodiment, targeted acoustic probing may be performed in a semi-invasive or invasive manner using an acoustic probe capable of producing focal acoustic pulses in conjunction with a semi-invasive or invasive instrument or procedure, such as a laparoscope, an endoscope, a remotely operable robotic instrument, a surgical instrument, or the like. Many such instruments are known in the art and would be amenable to use in association with intense focused ultrasound palpation methods and systems.

Another application of targeted acoustic probing of the present invention is the detection of dental caries. Acoustic probes having interfaces that effectively transmit ultrasound to tooth surfaces and internal tooth structures are used in this application. Such acoustic probes may have flexible interfaces that are capable of conforming to the surface conformations of teeth to provide positioning of the acoustic probe and application of intense focused ultrasound at various tooth locations. While incipient caries may not be painful absent targeted acoustic palpation, application of acoustic radiation forces to decayed teeth and tooth structures, is expected to evoke pain sensations or other sensations indicative of tooth decay. Diagnostic screening using acoustic palpation in the place of dental X-rays would reduce exposure to ionizing radiation and may provide more highly sensitive localization of tooth decay.

In yet another application, targeted acoustic probing as described herein is used to localize nerves and other sensitized tissues for guidance of needles or other delivery devices and delivery of anesthesia and/or analgesic and/or therapeutic agents to nerve sites and other sensitized sites. In this application, targeted acoustic probing may be applied to tissue sites in proximity to a nerve or another sensitized tissue site to identify the spatial location of nerves and sensitized sites. Because targeted acoustic probing may be administered non-invasively and is generally considerably less painful than the electrical probes (e.g., electrical needles) often used to locate nerve sites prior to administration of compositions, this application offers significant advantages compared to current nerve localization methods.

When the subject is conscious, the subject's subjective sensation of a particular biological response (e.g., pain) may be used for detection pain as the focus of the acoustic probe is moved within a generalized site. The subject's indication of pain may be recorded and automatically associated with the focal point of the acoustic palpation producing the response, and/or the spatial location of the focal point in the subject's body. When the subject is not conscious or his/her biological responses have been dulled or blocked, other physiological responses or indicia of biological responses are used to identify the source of pain. Exemplary biological responses to acoustic probing of tissues that may be detected and monitored to indicate responses of targeted tissue sites to acoustic stimulation are described above.

The subject's subjective perception of a biological response (e.g. pain) may be reported aurally to a clinician administering the intense focused ultrasound palpations, and the clinician notes the focal target site of the administered palpation to identify a source of the biological response (e.g. pain). Alternatively or additionally, the subject may indicate his or her perception of a biological response (e.g. pain) using an indicator that communicates with the system controller. In one embodiment, the subject may select from among a plurality of indicators to report his or her perception of a biological response (e.g. pain) at the time it occurs. The subject may report a response that's graded as to severity (e.g. 1-10 on a pain scale) and that indicates the type of sensation perceived. The subject's indication may be recorded and stored and may be automatically associated with the focal point of the intense focused ultrasound palpation producing the response, the correlative spatial coordinates of that focal point in the subject, the time of the response, measurements of other biological responses or subject physiological parameters at the time of the response, and the like.

Targeted acoustic probing of internal body sites may be performed in conjunction with and be guided by an imaging technique. An imaging modality may be used, for example, to examine broad investigational internal body sites and, in some embodiments, to target the acoustic probing to desired target sites within the investigational site. The imaging technique may also be used to visualize the targeted acoustic probing and to identify and/or map the target tissues and sites, to identify and/or map the sites evoking biological responses, and to identify and/or map target sites for application of therapeutic modalities. In addition to providing identification and/or mapping of sites evoking biological responses, identified sites may be marked (e.g., electronically) for follow-up, additional diagnostic evaluation, treatment, or the like. According to some embodiments, records (e.g., electronic record) preserving and identifying sites examined and/or marked during targeted acoustic probing may be generated for example, for follow-up, additional diagnostic evaluation, comparative purposes, or for treatment. The records preserving and identifying sites examined and/or marked during targeted acoustic probing may, in some embodiments, be compatible with auxiliary diagnostic and therapeutic systems so that the records may be transferred to and used directly in auxiliary imaging, diagnostic, and treatment systems.

Acoustic detection techniques that involve the application of acoustic interrogation signals to tissue site(s) and acquisition of acoustic scatter data are preferred for many applications. Alternative detection techniques, including near-infrared spectroscopy (NIRS), optical coherence tomography, computed tomography, magnetic resonance techniques including functional magnetic resonance imaging techniques, fluorography, radiography (e.g. X-ray) techniques, acoustic hydrophones and the like, may be used with target acoustic probing techniques of the present invention to examine internal body sites, to target acoustic probing to desired target sites, to target sites for application of therapeutic modalities, and for mapping and visualization purposes.

In systems of the present invention that implement ultrasound-based imaging and guided administration of intense focused ultrasound palpation pulses, separate imaging and intense focused ultrasound pulse generating probes may be used. In alternative embodiments, an integrated ultrasound probe is provided having both ultrasound imaging and intense focused ultrasound palpation capabilities. Imaging and palpation may be conducted sequentially and/or simultaneously.

Targeted acoustic probing of tissues is provided by the application of focused ultrasound pulses to the target tissue site. The level or type of biological response (e.g. pain) evoked may also be related to the magnitude, frequency, intensity, duration and/or pulse repetition rate of the focused acoustic palpation required to evoke the response. The applied acoustic radiation force is sufficient to induce a detectable biological response or sensation at damaged/sensitive tissue without producing medically undesirable changes in the examined tissue. For example, the acoustic radiation force applied must not produce shear forces in tissue of a magnitude sufficient to tear or damage tissue. The applied ultrasound, moreover, must not appreciably increase the temperature of examined tissue to the point of causing unacceptable damage, and it must not induce extensive or damaging cavitation or other sources of deleterious mechanical effects in the examined tissue. Suitable ultrasound dosages may be determined using well known techniques. For example, Fry et al. studied the threshold ultrasonic dosages causing structural changes in mammalian brain tissue and illustrate, in their FIG. 1, the acoustic intensity v. single-pulse time duration producing threshold lesions in white matter of the mammalian (cat) brain. Fry et al., Threshold Ultrasonic Dosages for Structural Changes in the Mammalian Brain, The Journal of the Acoustical Society of America, Vol. 48, No. 6 (Part 2), p. 1413-1417 (1970). Wright et al. (supra) have published experimentally determined threshold values of ultrasound parameters corresponding to the induction of pain for different ultrasound frequencies, stimulus durations and localizations of different types of tissue.

Additionally, the acoustic frequency must be low enough to penetrate tissue between the transducer and the target tissue site and high enough to produce a detectable biological response or sensation at damages/sensitive tissue. Within the parameters outlined above, higher frequency acoustic waves are more easily focused and, therefore, preferred. The intensity must be high enough to produce a sensation in target tissue, but not be so great as to induce undesirable changes in the examined tissue. The pulse length is preferably relatively short, but long enough to create a detectable sensation in the target tissue, as desired, while the pulse repetition frequency must be large enough to resolve medically interesting temporal features in the tissue, without inducing medically unacceptable changes in the tissue. An acoustic palpation may be administered as a single pulse or as pulse trains having varying numbers and frequencies of pulses.

Many protocols may be used to induce, and detect, a biological response (e.g. pain) at target tissue sites. In one protocol, intense focused ultrasound pulses having a first acoustic dose are applied to multiple target sites within a generalized site sequentially. If no biological response (e.g. pain sensation) is elicited by probing at any of the targeted sites, another series of intense focused ultrasound pulses having a second acoustic dose greater than the first are applied to multiple target sites sequentially to elicit a biological response. This protocol of applying intense focused ultrasound at progressively higher acoustic doses may be continued until a biological response is detected (or not). In another protocol, intense focused ultrasound pulses having progressively increasing acoustic doses (i.e. increasing intensity and/or duration and/or number or pulses and/or pulse repetition rate) may be applied sequentially to selected target sites until a biological response is detected (or not). In yet another protocol, targeted intense focused ultrasound having a first acoustic dose may be applied to a target site within a region of interest and a “control” site within a region believed to contain normal tissue of the same type. Control sites may be uninjured or unaffected contralateral sites for many tissues, or sites remote from the region of interest having similar physiological structures. In this protocol, target sites anticipated to produce biological responses are effectively compared directly to similar sites that are anticipated to be normal and would not produce biological responses until a threshold is attained.

As mentioned above, methods and systems of the present invention may also be used to determine a subject's pain sensitivity and threshold. Different individuals have different pain thresholds, and different types of tissue are more or less prone to evoke a pain response to an intense focused ultrasound palpation having a given acoustic dose. Because there are individual-specific and tissue-specific responses, methods of the present invention may include a preliminary subject- and/or tissue-specific evaluation or calibration performed prior to probing of target tissue sites to localize the sources of a biological response such as pain. In one embodiment, for example, a standardized pain threshold site may be palpated using intense focused ultrasound pulses of progressively increasing acoustic dose until a biological response (such as pain) is elicited. The subject's general pain threshold may then be taken into account in establishing a protocol for intense focused ultrasound palpation. In another protocol, tissue sites anticipated to be normal within different tissue types in a subject (e.g. finger tip, shin, arm and one or more internal organs) are acoustically probed to initially calibrate the system and, optionally, to assess the sensitivity of the subject to acoustic probing. Acoustic probing responses and values may be normalized from subject to subject using known techniques.

In another embodiment, a tissue-specific site having a physiological structure and tissue similar to that desired to be probed, such as tissue of the same type contralateral to inflamed or painful tissue, is assayed by applying intense focused ultrasound pulses of progressively increasing acoustic dose until a biological response (such as pain) is elicited. The subject's pain threshold in presumably normal, or undamaged, tissue of the type being assayed may then be taken into account in establishing a protocol for intense focused ultrasound palpation. Multiple probe heads may be used to probe control sites, such as standardized control tissues or presumed normal tissue, to determine subject-specific and/or tissue-specific pain thresholds.

In yet another embodiment, a subject's pain threshold may be evaluated with reference to both a generally superficial “normal” structure and a deep “normal” structure by applying intense focused ultrasound palpations having progressively increasing acoustic dose to both the superficial and deep normal structures. The subject's pain threshold may be determined using a combination, or ratio, of the pain thresholds of superficial and deep tissue structures. Many other protocols for evaluation a subject's pain threshold at various tissue sites may also be used. Pain threshold calibration may additionally be updated at intervals throughout a diagnostic or monitoring procedure.

Focused acoustic probing of tissue sites to localize physiological conditions and responses, such as pain, may be employed for any tissue sites where a sufficient acoustic window is available for application and passage of a focal acoustic beam. Localization of generally undifferentiated pain in the abdomen and/or pelvic area provides for diagnosis of appendicitis, cholecystitis, pancreatitis, numerous gastro-intestinal conditions and disorders characterized by pain, gall stones, kidney stones, cystitis and various painful bladder conditions, dysmenorrhea, ovarian and uterine conditions, and the like. Generalized, undifferentiated pain in the area of the spine and in other joints, such as the knee, ankle, shoulder, hip, sacroiliac, and other joints, may be localized using the focused acoustic probing techniques of the present invention, and the source of pain may be identified, for example, as cartilage, muscle, nerve, ligaments, tendons, and the like. Using focused ultrasound to induce acoustic palpation, for example, back pain may be localized and identified as disc-related, or as originating in the facet, vertebral body, nerve, muscle or the like. Peripheral nerve-related pain and lymphadenopathies resulting, for example, from cancer and infections, may also be diagnosed and localized. Methods and systems of the present invention may also be used to monitor the condition and health of tissue following an intervention or course of therapy and to monitor and evaluate the healing process.

Methods for assessing a subject with chronic pain, for diagnosing chronic pain disorders, and for assessing the severity of chronic pain disorders are also contemplated. In one embodiment, methods for assessing a subject with chronic pain involve selecting at least one localized target site internal to the subject's external body surface and within an area of sensitized tissue; applying intense focused ultrasound pulses having different acoustic doses to the localized target site; and assessing the duration and/or intensity and/or quality of biological responses (e.g., pain) to the focused ultrasound pulses. Mapping of pain responses, and evaluating the scope and severity of pain responses may be performed by application of focal ultrasound palpations of an appropriate magnitude, frequency, intensity, duration and/or pulse repetition rate to a plurality of localized target sites within an area of sensitized tissue. Comparison of the intensity and/or duration and/or quality of responses to iFU protocols targeted to sensitized tissue sites of patients suffering from undifferentiated chronic pain and from chronic pain disorders with the intensity and/or duration and/or quality of responses to the same or similar iFU protocols targeted to the same anatomical tissue sites of “normal” subjects may be performed to assess, or to quantify, the severity of patients' pain, the progression or amelioration of pain, the success or failure of treatment regimen, and the like.

### BRIEF DESCRIPTION OF THE FIGURES

FIG. 1 is a schematic diagram illustrating a system of the present invention for inducing a sensation of pain, or another biological response to a generally focused ultrasound pulse and incorporating an ultrasound imaging system for imaging, visualizing and/or mapping the results.

FIG. 2 is a schematic diagram illustrating an ultrasound probe of the present invention for palpating tissue by applying intense focused ultrasound and incorporating an ultrasound imaging capability.

FIG. 3 is a schematic diagram illustrating exemplary system components for applying intense focused ultrasound (iFU) and for imaging, visualizing and/or mapping the results and identifying target tissues eliciting a biological response to acoustic probing, and interfacing with a secondary diagnostic or therapeutic device.

FIG. 4A is an image illustrating a side view of an experimental intense focused ultrasound palpation probe of the present invention.

FIG. 4B is an image illustrating a side perspective view an experimental u/s palpation probe of the present invention.

FIG. 5 is a schematic diagram illustrating a system of the present invention comprising an intense focused ultrasound palpation probe and a controller.

FIG. 6 is a schematic diagram illustrating another embodiment of a system of the present invention comprising an ultrasound probe having both intense focused ultrasound palpation and imaging capabilities, a controller for image guided probing, and a subject response indicator.

FIG. 7 is a schematic diagram illustrating another embodiment of a system of the present invention for administering intense focused ultrasound palpations using a plurality of probes.

FIG. 8 illustrates experimental results demonstrating the acoustic dose necessary to produce an unambiguous withdrawal response after application of iFU to sensitized and normal paws.

FIG. 9 shows the results of testing for pain sensitivity and threshold in human subjects using intense focused ultrasound palpation.

### DETAILED DESCRIPTION

It will be appreciated that the methods and systems of the present invention may be embodied in a variety of different forms, and that the specific embodiments shown in the figures and described herein are presented with the understanding that the present disclosure is considered exemplary of the principles of the invention, and is not intended to limit the invention to the illustrations and description provided herein. It will also be appreciated that while many embodiments are described with reference to the localization of pain responses, many other types of biological responses may be induced, and localized, using methods and systems of the present invention. It will be appreciated, moreover, that while the use of therapeutic ultrasound modalities is disclosed specifically, many different types of therapeutic modalities may be used to treat a subject at target sites identified using the acoustic palpation techniques described herein.

Systems of the present invention comprise at least one ultrasound probe head comprising an ultrasound transducer, a plurality of ultrasound transducers, or one or more ultrasound transducer array(s) and incorporate, or communicate with, an ultrasound signal generator, an optional signal amplifier and a controller. In a simplified embodiment, an ultrasound probe head may be provided that is capable of producing intense focused ultrasound pulses of a predetermined pulse duration, intensity, pulse repetition rate, or the like, at a predetermined focal point at some distance from the probe head. In another embodiment, an ultrasound probe head is capable of producing intense focused ultrasound pulses at a selectable focal point at a selectable distance from and spatial orientation with respect to the probe head. In yet another embodiment, an ultrasound probe head may be provided that is capable of producing intense focused ultrasound pulses having variable pulse duration, intensity, pulse repetition rate, and the like, with one or more of the variable parameters selectable by an operator.

Multiple transducers and/or transducer arrays may be provided in an integrated probe head, or in multiple probe heads, and used to produce intense focused ultrasound pulses having different foci and/or pulse intensity and/or other properties. Multiple transducers and/or arrays producing intense focused ultrasound pulses may be used sequentially and/or in combination. Multiple probe heads having different geometries and/or different intense focused ultrasound pulse capabilities may be provided for use in interfacing with different body surfaces and probing different internal sites. Multiple probe heads may interface with a common controller, sequentially or simultaneously. Selectable target pulse parameters, such as pulse focus, pulse duration, pulse intensity, pulse magnitude and/or pulse repetition rate may be controllable from the probe head and/or from the controller.

In another embodiment, an imaging device is provided that is capable of imaging a generalized site prior to use of the probe. An imaging ultrasound probe may be provided separately from or in combination with an intense focused ultrasound targeting probe, and imaging may be used to actively or passively guide the application of intense focused ultrasound pulses. In one embodiment, for example, an operator may view an image (e.g. an ultrasound diagnostic image) of a generalized site and may select target sites within the generalized site for intense focused ultrasound palpation. Other types of guidance systems may be used to image, or to identify, desired target sites and/or sites eliciting biological responses. Light-based systems, including NIRS and laser-based systems may be used for guidance, for example, and may be provided separately from or in combination with an intense focused ultrasound targeting probe.

In one embodiment of an integrated imaging and palpation system, the operator may “mark” desired target sites on an image display and instruct the palpation probe to apply intense focused ultrasound pulse(s) having predetermined or selectable properties to the marked target sites to acoustically palpate the desired target sites to localize and grade biological responses. In another embodiment, the operator may “mark” or indicate desired target sites and instruct the system to perform one or more acoustic palpation routines to localize and grade biological responses. In yet another embodiment of an integrated system, an operator may view an image of a generalized site and visualize the intense focused ultrasound pulse spatial location overlaid over the generalized site image to spatially locate target palpation sites and biological responses.

Controllers used in conjunction with ultrasound palpation probes of the present invention may be adapted for processing, recording, storing, and/or displaying data. In one embodiment, various selectable ultrasound palpation routines are pre-programmed or programmable into the system and an operator may select one or more routines and apply them to selected target palpation sites. Ultrasound palpation routines may involve application of intense focused ultrasound pulses of ascending and/or descending intensity, amplitude and/or pulse duration, for example.

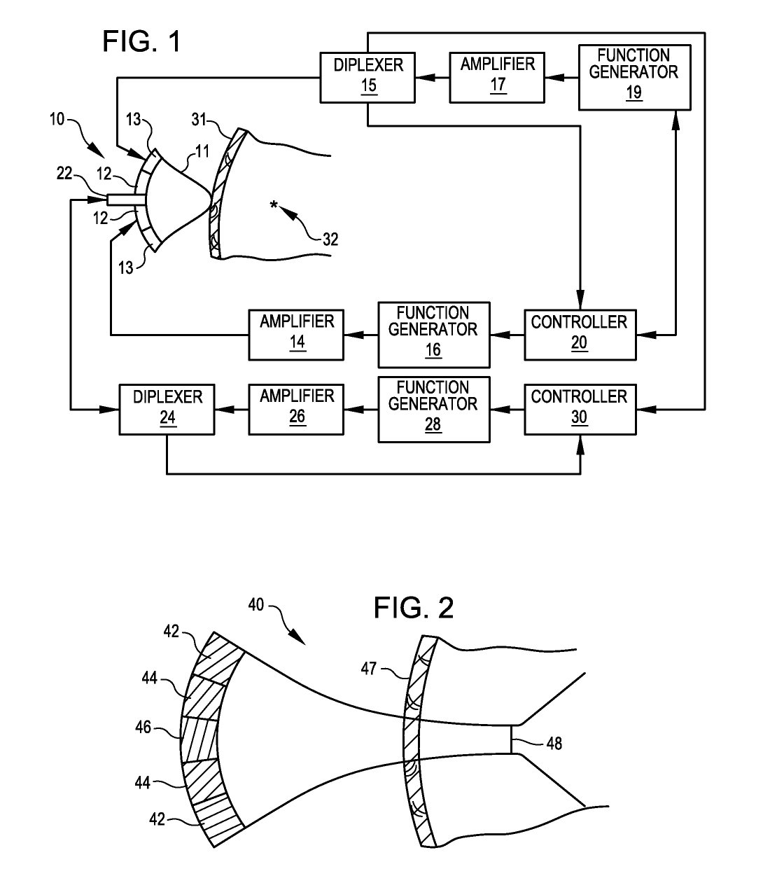

FIG. 1 is a schematic diagram illustrating one embodiment of a system of the present invention comprising an acoustic probe and driving and control systems for non-invasively palpating tissue using intense focused ultrasound pulses. Acoustic probe 10 comprises one or more acoustic source(s) 12 for generating an acoustic radiation force at a at a distance from the transducer(s) and probe head 11. The acoustic probe includes a probe “head” 11 constructed from an acoustically transmissive material and providing spatial separation between the acoustic source(s) and the body surface 31. The internal space between the acoustic source(s) and the probe head is generally filled with an acoustically transmissive material such as a gel or liquid, or the internal space may be composed of a solid, acoustically transmissive material.

According to one embodiment, an external surface of probe-head 11, or a contact external surface forming a portion of the external surface of probe-head 11 is constructed from a substantially liquid- and gel-impermeable material, while the internal space of the probe-head, underneath the external surface, is filled with a gel-like material or liquid or other malleable material that provides efficient acoustic transmission and also allows the probe-head to assume different configurations and closely contact surfaces having different conformations, shapes, textures, and the like, thereby providing good acoustic coupling between the probe head and objects or surfaces to which acoustic palpations are administered. The outer contour of the probe-head may be curved, as shown, or angular and may have a generally conical shape with a flat or curved interface portion. Alternatively, the outer contour of the probe-head may be substantially flat and planar, or may have a variety of curved or angular conformations.

Acoustic source(s) 12 are driven by and operably connected to an amplifier or power source 14, which is operably connected to one or more function generator(s) 16, which is operably connected to a controller 20. Controller 20 preferably has the capability of data acquisition, storage and analysis. While these components are illustrated separately, it will be appreciated that this illustration is merely schematic and one or more of these functions may be housed in an integrated device, and that additional functions, controllers, and the like may be provided. Controller 20, function generator 16 and amplifier 14 drive acoustic source(s) 12 at a desired frequency, intensity and pulse repetition rate in an acoustic radiation force mode to administer focal, generally high intensity ultrasound pulses to target tissue sites to produce a biological response, such as a sensation of pain, at tissue target site 32 without producing undesired side effects. The operating acoustic parameters are related to one another and suitable operating parameters are described below and may be determined with routine experimentation.

Acoustic probe 10 may additionally comprise a second acoustic source 13 driven by and operably connected to a diplexer 15, which is operably connected to an amplifier or power source 17, which is operably connected to a function generator 19, which, in turn, communicates with controller 20. In the embodiment illustrated, the two acoustic sources 12, 13 are controlled by a common controller 20. Multiple ultrasound sources (transducers) may be operated independently of one another to provide intense focused ultrasound palpations at different target sites, or multiple acoustic transducers may be operated in a coordinated fashion to produce a tissue palpation or displacement at a desired target tissue site at their mutual focus, shown as target site 32 in FIG. 1.

Systems for palpating tissue to identify and localize the source of sensations such as pain may also incorporate a targeting system for targeting the acoustic palpations to the desired target tissue site and for locating the target tissue sites tested and producing responses. The targeting and localization system may be an acoustic (ultrasound) system, or it may employ an alternative modality, such as magnetic resonance, computed tomography, nIR spectroscopy, or the like. In many embodiments, the intense focused ultrasound palpation system comprises an imaging system that provides real-time visualization of the focal acoustic palpations and the anatomical structures and precise location of the target sites. In one embodiment, also illustrated in FIG. 1, a diagnostic ultrasound probe 22 is incorporated in the acoustic source probe 10 for imaging larger areas surrounding target palpation sites and visually localizing the target palpation sites within the larger areas. The diagnostic ultrasound probe may be also be used for selecting focal acoustic target palpation sites. Diagnostic imaging probe 22 is in operable communication with diplexer 24, amplifier 26, function generator 28 and controller 30 for generating diagnostic imaging ultrasound pulses and receiving acoustic data. Controller 30 may also interface with controller 20 and other electronics systems operating the acoustic sources. In preferred embodiments, a monitor may be provided for displaying and visualizing both the larger interrogation area and the target palpation sites within the area.

FIG. 2 illustrates one embodiment of an acoustic source and probe combination 40 that is suitable for use in systems of the present invention. Source and probe combination 40 comprises confocal, annular acoustic sources 42 and 44 and a diagnostic ultrasound imaging probe 46. Phasing acoustic sources 42 and 44 at slightly different frequencies produces a significant radiation force only at their mutual focus, indicated underneath tissue surface 47 at focal target site 48. The radiation force produced at the focal target site, at certain acoustic doses, produces a subjectively detectable sensation or an objectively detectable biological response in the tissue.

The acoustic dose may be adjusted to produce a biological response and/or sensation at focal target site 48. When a single acoustic source is used, or the sources are used such that there is no difference in frequency between the sources, the result may be a unidirectional palpation of the tissue at the target location that coincides with the overlapping foci, with a negligible oscillatory component for the duration of each acoustic pulse. This tissue palpation may also produce a biological response and/or sensation at the focal target site.

An acoustic source and probe combination comprising one or more acoustic sources may also be used, in combination with an imaging system that employs an imaging modality other than an acoustic imaging modality, to acoustically stimulate or palpate tissue at target sites to localize tissue responses to the focused ultrasound, such as pain. The acoustic source and probe combination may be used in combination with an ultrasound imaging system, as described above, to provide visualization and/or mapping of target site(s) and aid targeting of the acoustic radiation forces and localization of biological responses, such as pain. The imaging system may, additionally or alternatively, employ a tissue imaging modality other than an ultrasound modality, such as magnetic resonance imaging (including functional magnetic resonance imaging), computed tomography (CT), optical coherence tomography (OCT), near infrared optical detection techniques (e.g., NIRS), fluoroscopy, radiography (e.g., X-rays) or the like. This alternative tissue imaging modality may be provided in a separate system having separate control functions, but is preferably integrated or integratable with an ultrasound system for administering intense focused ultrasound as described herein.

An acoustic source and probe combination comprising one or more acoustic sources may also be used in combination with a therapeutic or treatment modality that employs an ultrasound-based treatment system for administering therapeutic ultrasound (e.g., tissue warming ultrasound, HIFU ultrasound, and/or generally high or low intensity acoustic shock wave ultrasound). For methods and systems involving administration of therapeutic ultrasound, a therapeutic ultrasound source may be integrated with an ultrasound source and detector device providing acoustic palpation, as described herein, and/or providing ultrasound imaging capability. Ultrasound probes and arrays providing both diagnostic and therapeutic ultrasound capabilities are known in the art and may be used, and/or modified, to additionally provide acoustic palpation and tissue targeting and identification as described herein. In some embodiments, a single ultrasound probe, or an ultrasound array, may be operated and controlled to provide imaging of tissue, palpation of selected target sites within tissue, mapping of the palpated target sites within tissue and identification of palpated target sites eliciting a subjective response (e.g. pain) or a detectable biological response (e.g. heart rate; blood flow, pressure, composition or the like; electrical heart or brain activity; or the like), and ultrasound treatment of selected target sites within tissue. In alternative embodiments, multiple ultrasound probes, or multiple ultrasound arrays, or a combination of one or more ultrasound probe(s) and one or more ultrasound array(s) may be operated and controlled to provide imaging, palpation, mapping, identification and treatment.

In another aspect, systems and methods of the present invention may be employed for targeting of a diagnostic and/or therapeutic device. In this aspect, tissue palpation, targeting, identification and/or localization techniques of the present invention may be used in conjunction with, or integrated with, various types of diagnostic devices, such as biopsy devices, endoscopic and laparoscopic devices, catheter-based devices and other types of minimally invasive devices, as well as with surgical and minimally invasive surgical devices, including robotic, radio-surgical and catheter-based devices. Systems and methods of the present invention providing acoustic (ultrasound) target palpation, identification and/or localization may also be used to monitor target sites and tissues following the administration of a diagnostic or therapeutic modality.

Treatment modalities other than ultrasound treatment modalities may also be employed to provide treatment to target sites identified using acoustic palpation techniques of the present invention, and treatment modalities of various types may be integrated with acoustic (ultrasound) target palpation, identification and/or localization techniques of the present invention. Systems and methods of the present invention may thus, additionally or alternatively, employ a tissue treatment modality other than an ultrasound modality, such as an ablative modality (e.g., thermal and non-thermal tissue ablation techniques such as RF ablation, cryo-therapeutic ablation, electrolytic ablation, and the like), targeted administration of a therapeutic agent (e.g., a drug or biological agent, combination of agents, radioactive agent, and the like), and other therapeutic modalities. The tissue treatment modality may be provided in a separate system having separate control functions, or may be integrated or integratable with an ultrasound system for administering intense focused ultrasound as described herein. According to another embodiment, auxiliary diagnostic and treatment systems may use, as input, target identification(s) and data acquired using acoustic palpation systems of the present invention.

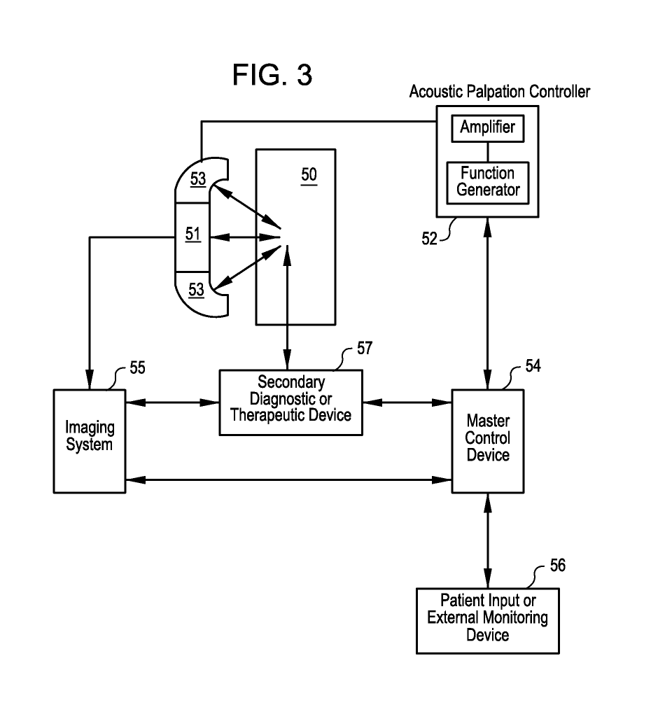

FIG. 3 shows a highly schematic diagram illustrating various components comprising systems of the present invention, and auxiliary components or devices that may be interfaced with acoustic palpation systems of the present invention. Tissue under examination is represented by the central rectangular box 50. An imaging system 55 preferably drives, and controls acquisition of target data using imager 51 during an acoustic palpation operation. Acoustic palpation controller 52 comprising, e.g., an amplifier and a function generator, drives and controls application of acoustic palpation pulses through acoustic source and/or detector 53. Both the imaging system and the acoustic palpation controller may operate under the control of master control device 54. In alternative embodiments, the imaging system and acoustic palpation controller may be integrated and/or housed centrally in a master control device. A patient input and/or external patient monitoring device 56 may interface both with the patient and with the master control device 54. Similarly, secondary diagnostic or therapeutic device(s) represented schematically as 57 may interface both with the patient and with the master control device.

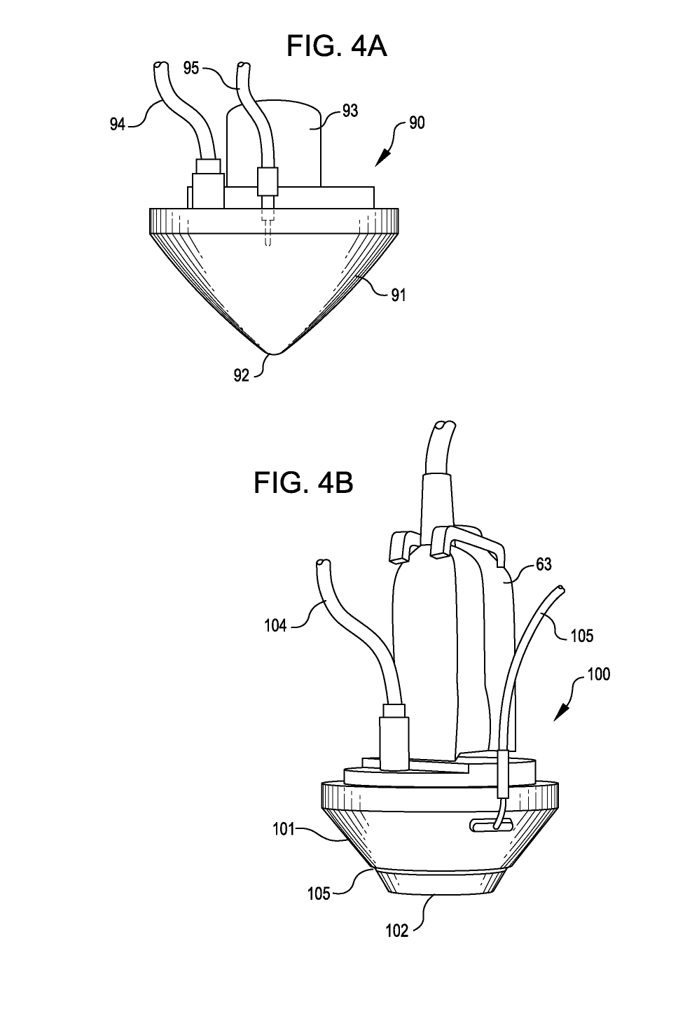

FIG. 4A illustrates an experimental intense focused ultrasound palpation probe 90 of the present invention. In this embodiment, annular transducers are housed in a housing structure 91 having a generally cone-shaped configuration. It will be appreciated that many other housing structure configurations may be provided. The housing structure may be constructed as a substantially rigid coupling cone, comprising a solid material having high acoustic transmission properties. Alternatively, housing structure 91 may comprise an outer structure constructed from a solid material having high acoustic transmission properties and substantially impermeable to liquids, in combination with an inner cavity substantially filled with an acoustically transmissive material such as a gel or a liquid. The terminal end 92 of the housing structure coupling cone 91 may be generally flattened or provided with a gently curved surface for contacting and interfacing with an external tissue site on a subject, and for providing a suitable acoustic field of view. The focal point(s) of the acoustic transducer(s) is generally located at some distance beyond the terminal end of the coupling cone 91 and may be fixed or adjustable. A support structure 93 may be provided, as indicated, to facilitate handling and positioning of the intense focused ultrasound palpation probe. Suitable electrical connections 94 are provided for driving the transducer(s), and conduit(s) 95 may be provided for supplying transmissive material to or evacuating material from an internal cavity of the housing structure coupling cone, as illustrated.

FIG. 4B illustrates another embodiment of an experimental intense focused ultrasound palpation probe 100 of the present invention. In this embodiment, one or more ultrasound transducers are housed in a housing structure 101 having a generally cone-shaped structure. It will be appreciated, as with the previous embodiment, that many other configurations of housing structures may be provided. The housing structure 101, shown as a coupling cone, may comprise an outer substantially solid structure having high acoustic transmission properties enclosing an inner cavity substantially filled with an acoustically transmissive material such as a gel or a liquid. The terminal end 102 of coupling cone 101 is generally flattened for interfacing with an external body site on a subject and providing an appropriate acoustic field of view. The focal point(s) of the acoustic transducer(s) is generally at some distance beyond the terminal end 102 of the coupling cone and may be fixed or adjustable.

Ultrasound palpation probe also incorporates an imaging ultrasound transducer 63 mounted generally in a central probe location for imaging target regions and sites. Suitable electrical connections 104 are provided for driving the transducers, and conduit(s) 105 may be provided for supplying transmissive material to or evacuating material from the internal cavity of the housing structure.

In one embodiment, the acoustic probe itself may be mechanically adjustable to change the focal point of the ultrasound palpation transducer(s). Mechanical probe housing 101 as shown in FIG. 4B is configured, for example, to effectively change the target position of a focal acoustic palpation pulse by changing the distance between the transducer and a target site. The terminal end 102 of probe housing 101 telescopes and retracts at groove 105 to change the location of the probe head 62 with respect to the ultrasound palpation transducer(s). The probe housing 101 illustrated in FIG. 4B is in a retracted, smaller dimension probe head position and is extendable, at groove 105, to position terminal end 102 a distance from the remainder of probe housing 101, thereby changing the distance between the transducer and the target site and changing the target position of the focal acoustic palpation pulse. In another embodiment, a terminal end of the probe may be movable to an extended or retracted position using electrical or electronic mechanisms. In yet another exemplary embodiment, the probe housing may have an inflatable member or resizeable component that may similarly function to change the location of the terminal portion of the probe head with respect to the ultrasound palpation transducer(s) and thus provide multiple foci targeting.

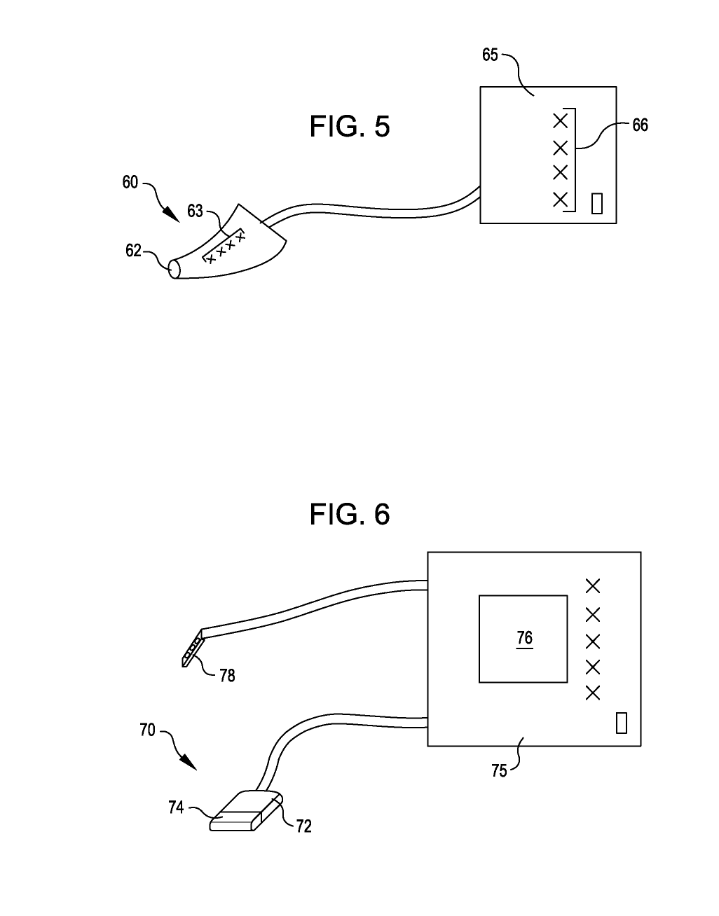

FIG. 5 shows a schematic diagram illustrating an acoustic palpation probe 60 having a probe head 62 for contacting a body surface, either directly or indirectly through an acoustic coupling material or structure. The acoustic probe 60 is in communication with a device controller 65 providing power to probe 60 and, optionally, providing selectable control of the acoustic palpation parameters. In one embodiment, probe 60 has a fixed focal point and selectable controls 63 provided on the probe body and/or selectable controls 66 provided on controller 65 may be actuated by an operator, or adjusted, to provide a desired acoustic dose, acoustic intensity, pulse duration, etc., for an acoustic palpation protocol. Alternatively, predetermined acoustic palpation protocols may be programmed or programmable in controller 65 or probe 60. Multiple probes having different focal points, configurations, operating parameters and capabilities, and the like, may be interfaced with a common controller 65. Probe 60 may optionally incorporate an imaging device providing visualization of a target area and guided targeting of the acoustic palpation pulses.

FIG. 6 shows a schematic diagram illustrating an acoustic palpation probe 70 having a probe head 72 comprising at least one acoustic transducer and/or transducer array for producing focal acoustic palpations. Probe 70 also preferably incorporates an imaging device, such as an ultrasound imaging scan head, for visualizing a target region and guiding the administration of intense focused ultrasound palpations. Images of a target region may be displayed on a display 76 integrated with controller 75, or provided separately.

Probe head 72 is configured for contacting a body surface, either directly or indirectly through an acoustic coupling material or structure. Acoustic coupling component 74 contacts probe head 72 and facilitates acoustic coupling of the probe head 72 to the surface (e.g. body surface) to be contacted. Acoustic coupling component 74 comprises an acoustically transmissive medium such as a gel or a liquid, generally enclosed in a liquid impermeable covering. The acoustic coupling component may be permanently or transiently mounted on probe tip 72 during a palpation protocol. In one embodiment, acoustic coupling component 74 may be provided as a disposable or reusable “packet” mountable in or on the surface of probe tip 72 using an adhesive, a mechanical or magnetic mounting system, or the like. Acoustic coupling components having different configurations and dimensions may be provided.

The acoustic probe 70 is in communication with a device controller 75 providing power to probe 70 and, optionally, providing selectable control of the acoustic palpation parameters. In one embodiment, probe 70 has a fixed focal point and, in another embodiment, probe 70 comprises multiple transducers or transducer arrays providing different focal points. Selectable controls provided on the probe body and/or on the controller 75, may be selected by an operator, or adjusted, to provide operation of a selected or multiple transducers to provide a single or different intense focused ultrasound pulses, to provide desired acoustic dose(s), acoustic intensity(ies), pulse duration(s), etc., for various acoustic palpation protocols. Alternatively, predetermined acoustic palpation protocols may be programmed or programmable in controller 75 or probe 70. Multiple probes having different focal points, configurations, operating parameters and capabilities, and the like, may be interfaced with a common controller 75.

The system illustrated in FIG. 6 also includes an indicator device 78 that interfaces with controller 75 and is operated by a subject to provide feedback on biological responses (e.g. sensations) evoked during focal acoustic palpation of target sites. In one embodiment, for example, indicator device 78 may be used by a subject to indicate, and/or to grade a pain response to focal acoustic palpation. In another embodiment, indicator device 78 may be used to indicate the type of biological response, e.g. sensation, evoked by focal acoustic palpation. Indicators may be provided as selectable, mechanically or electrically operated “buttons” on device 78, or indicators may be provided and visualized on a touch screen device or using other peripheral devices that are well known in the art. The indicator device preferably interfaces with controller 75 to record a subject's responses to administration of focal acoustic palpation. The subject's responses may be associated with the intense focused ultrasound palpation protocol to identify the target site, acoustic dose, time, etc. evoking a biological response.

Controller 75 may have data processing, recording, storage, and display capacities, and may also be capable of interfacing with another device, such as a computer system or an integrated medical records system. In some embodiments, as mentioned previously, the controller may provide integration of the imaging and palpation systems, such that acoustic palpation may be targeted to desired sites by identifying target sites on an image and, likewise, target sites eliciting a biological response (e.g. pain) may be identified and displayed on the image and automatically correlated with spatial coordinates and physiological structures corresponding to those spatial coordinates in the subject's body. In additional embodiments, a controller may provide integration and automation of the imaging and palpation systems, such that the controller administers acoustic palpation protocols, records and integrates a subject's responses (or biological responses) and modifies acoustic palpation protocols and tissue targets based on the feedback until target site(s) at which desired sensations and/or biological responses are induced are positively identified and localized with the desired specificity.

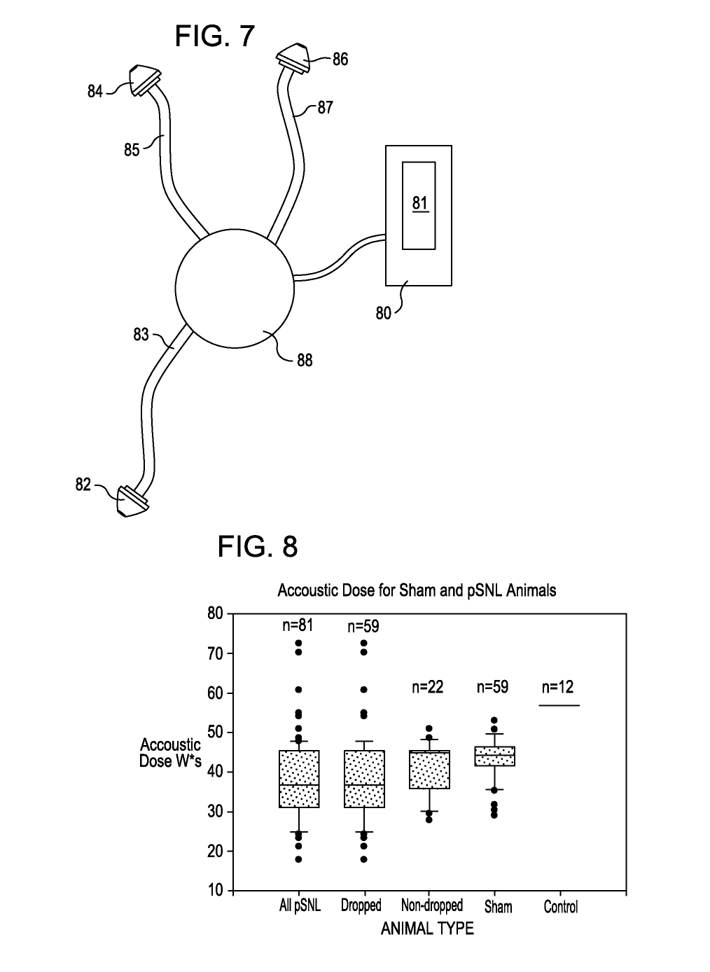

FIG. 7 illustrates yet another exemplary embodiment of an integrated system of the present invention comprising multiple acoustic probes 82, 84, 86 mounted on a common intermediate 88 in communication with controller 80. One or more of the multiple acoustic probes may comprise an imaging probe providing visualization of a target region on a display 81. Multiple acoustic probes may have similar or different foci and acoustic palpation properties and may be used independently of one another to probe multiple target sites, or to provide different intense focused ultrasound pulses to a common target site. In another embodiment, the operation of multiple acoustic probes may be coordinated to provide an intense focused ultrasound palpation at a target site where two or more pulses converge.

The multiple acoustic probes 82, 84 and 86 are preferably mounted on adjustable positioning devices, such as positioning arms 83, 85, 87, facilitating placement of the probes on different interrogation sites on the subject simultaneously or sequentially. The arms may be adjustable by means of multiple segments and pivot points, or they may be substantially continuously adjustable using, for example, gooseneck-type conduits or sections. Boom-type mechanical devices may additionally or alternatively be used to provide accurate probe positioning. In one embodiment, the positioning device(s) exert a pressure in the direction of the probe face following positioning to bias the probe face toward the body surface and ensure positive contact of the probe face with the body surface during an acoustic palpation protocol.

In one embodiment, detachable, exchangeable probe-heads having different acoustic imaging, palpation and/or treatment capabilities, providing different foci and/or different fields of view, different configurations and the like, may be provided and operably attached to and detached from a common handle section that communicates with a controller and, optionally, with other devices. Communication between detachable probe-heads, handles and controller components may be provided using wired or wireless technologies.

Commercially available components may be used in systems of the present invention. The following description of specific components is exemplary, and the systems of the present invention are in no way limited to these components. Experimental systems for administering intense focused ultrasound pulses to elicit pain responses are also described in the examples, below. High intensity focused ultrasound transducers that are suitable for intense focused ultrasound palpation are available from Sonic Concepts, Woodinville, Wash. Multi-element transducers have been used by researchers and are described in the literature. A multiple focused probe approach for high intensity focused ultrasound-based surgery is described, for example, in Chauhan S, et al., Ultrasonics 2001 January, 39(1):33-44. Multi-element transducers having a plurality of annular elements arranged, for example, co-axially, are suitable. Such systems may be constructed by commercial providers, such as Sonic Concepts, Woodinville, Wash., using technology that is commercially available. Amplifiers, such as the ENI Model A-150, are suitable and are commercially available. Diplexers, such as the Model REX-6 from Ritec, are suitable and are commercially available. Function generators, such as the Model 33120A from HP, are suitable and are commercially available. Many types of controllers are suitable and are commercially available. In one configuration, a Dell Dimension XPS PC incorporates a Gage model CS8500 A/D converter for data acquisition, and utilizes LabView software from National Standards for data acquisition and equipment control. In some embodiments, an ATL transcranial Doppler probe, Model D2TC, is used for detection.

Diagnostic ultrasound imaging systems may also be used to generate acoustic signals that produce acoustic palpations for use in methods of the present invention. Diagnostic ultrasound imaging systems that generate signals with carrier frequencies generally greater than about 0.5 MHz and less than 100 MHz and are adapted to administer two or more pulses having individual durations of from about 0.01 μsec to about 50 msec, with pulse repetition frequencies in the range of from about 0.01 Hz to about 1000 Hz and pulse intensities in the range of from about 1 W/cm² to about 10,000 W/cm² may be suitable for use in acoustic palpation for various applications as described herein. This means that existing diagnostic ultrasound imaging systems having the capabilities described above may be modified, or retrofit, to provide the additional functionality of performing targeted acoustic probing according to the present invention. According to one exemplary embodiment, ultrasound probes having specialized configurations and/or features suitable for targeted acoustic probing may be interfaced with operated by diagnostic ultrasound imaging systems that are modified to administer ultrasound pulses for targeted acoustic palpation. Any of the device controllers described and illustrated above may thus comprise a diagnostic imaging system, or components of a diagnostic imaging system having the capabilities described herein.

The variables that govern intense focused ultrasound for detection and localization of biological responses such as pain are intensity (W/cm²), dose (W/cm²)*sec, frequency (Hz), pulse length (seconds), number of pulses, and pulse repetition frequency (Hz). The generally high energy intense focused ultrasound pulses used to palpate target tissue are generally considered HIFU pulses. One measure of ultrasound action is intensity I, in units of Watts/square cm=W/cm² of ultrasound emitted by a device. Two different measures of intensity are used. The spatial peak and temporal peak intensity (I_sptp) is relevant for short-pulsed devices and provides a measure of the most intense portion of ultrasound generated by a device. The following calculation may be used to derive (I_sptp): (I_sptp)=(P²)/2*rho*c, where the pressure (p) generated by the device is measured, rho is the density and c is the sound speed. In general, the acoustic intensity generated by focused ultrasound of the present invention measured as I_sptp is desirably less than about 10⁹ W/cm² per pulse and, for many embodiments, is less than about 10⁷ W/cm² per pulse and, in yet other embodiments is less than about 10⁶ W/cm² per pulse. Another measure of acoustic intensity is given by its spatially averaged and temporally averaged value (I_sata). In general, the acoustic intensity generated by focused ultrasound of the present invention measured as I_sata is desirably less than about 10⁴ W/cm² per second of exposure to the intense focused ultrasound and, for many embodiments, is less than about 10³ W/cm² per second of exposure to the intense focused ultrasound and, for yet other embodiments, is less than about 10² W/cm² per second of exposure.

Suitable ultrasound parameters of focused ultrasound pulses for palpation of tissue as described herein include the following: center or carrier frequency emitted by the transducer f_{—}0=from about 0.1 MHz to about 30 MHz, generally from about 0.9 to 8 MHz, and often from about 1.0 to 3.0 MHz; I_sata (spatial average, temporal average intensity) ranges from about 0.5 to 5000 w/cm², in some embodiments from about 5 to 1000 w/cm², in some embodiments from about 10 to 500 w/cm² and, in yet other embodiments, from about 20 to 100 w/cm². The acoustic dose, calculated as the I_sata multiplied by the duration of the ultrasound pulse, ranges from about 1-1000 (W/cm²)*sec, in some embodiments from about 1-100 (W/cm²)*sec, in some embodiments from about 1-60 (W/cm²)*sec, and in yet other embodiments, from about 5 to 30 (W/cm²)*sec.

The duration of individual palpation interrogation pulses may range from about 0.0001 to about 10 seconds, in some embodiments from about 0.01 to 1 second, and in some embodiments from about 0.1 to 0.5 second. The time between individual palpation interrogation pulses is sufficient to prevent heat accumulation in the tissue targeted for palpation and may generally be from about 0.01 to about 120 sec, in some embodiments from about 0.1 to about 60 sec, and in yet other embodiments from about 0.5 to about 30 sec. Suitable duration periods for individual palpation interrogation pulses and the time between individual pulses depends, to a large degree, on the acoustic intensity and acoustic dose of the pulses being administered, and on the type of tissue targeted. Different tissue types have different sensitivities to acoustic palpation. Practitioners in the art may determine suitable pulse intensities, acoustic doses, durations, repetition rates, and the like using routine experimentation.

Individual intense focused ultrasound acoustic palpation pulses may be used, as described above and below in the experimental results, to detect and/or spatially locate tissue targets inducing various biological responses, and for the other applications described herein. Multiple pulses and pulse trains may also be used to detect tissue targets eliciting biological responses. The pulse repetition frequency for acoustic palpation using multiple pulse trains is generally in the range of from about 1-20 Hz.

All of the citations and publications described herein, including patents and non-patent publications, are hereby incorporated herein by reference in their entireties.

The following examples are offered by way of illustration and are not intended to limit the invention in any fashion.

### Example 1

A prototype image-guided intense focused ultrasound palpation device was constructed, as illustrated in FIG. 4B. It consisted of a high intensity focused ultrasound (HIFU) transducer coupled with a diagnostic ultrasound probe from an Acuson diagnostic ultrasound device. The prototype device was used by the investigator to generate transient sensations in normal tissue in the palm of his hand using short, sharp but energetically small bursts of ultrasound. The following acoustic protocol evoked transient sensations of pain: a single pulse of 10 ms in duration at a frequency of 1.1 Mhz and spatial peak, time average intensity of approximately 10 W/cm². The investigator did not perceive any lasting effects of the ultrasound application.

### Example 2

Experimental studies were conducted in an animal model to evaluate whether probing a sensitive tissue with intense focused ultrasound (iFU) produced detectable sensitivity. The prototype ultrasound transducer device consisted of a commercial piezo-electric, flat transducer built into a solid, cylindrical cone shaped aluminum housing having a flat distal face. The dimensions of the housing allowed ultrasound emitted from the transducer to have its focus at the proximal tip of the aluminum housing. The focus of the device was characterized with a needle hydrophone to measure the spatial peak and temporal peak intensity (I_sptp) as described in Miao et al. (2005). The focus of the experimental iFU device was about the size of a grain of rice, extending less than a centimeter from the transducer head with a width of less than half a centimeter onto and into the adjoining tissue. It was not necessary to provide image guidance of the focused ultrasound device, since the focal point for acoustic palpation was fixed and known.

The solid cone device was driven by two function generators (33120A, Hewlett Packard/Agilent, Palo Alto, Calif.) and an amplifier (A150 RF Power Amplifier, ENI, Rome, Italy). The first function generator's role was to gate the pulse to a specific duration. The second function generator, in series with the first, was used to modify the acoustic output and ensured that the pulse was emitted at a specific frequency. The amplifier increased the signal amplitude from the function generators and sent it to the solid cone device. An oscilloscope (Wave Runner LT 322, LeCroy, Chesnut Ridge, N.Y.) measured the duration of the pulse, its carrier frequency, and the acoustic intensity delivered by the iFU device.

Individual pulses of ultrasound were used in the experimental protocol. The center frequency was 1.15 MHz, with pulse durations of 0.1, 0.2, 0.33 and 0.5 seconds. The mechanical force exerted by the pulses was measured with a force balance as described in Poliachik et al. (2001). From these measurements the spatial average and temporal average intensities (I_sata) as well as the acoustic doses (I_sata multiplied by ultrasound pulse duration) were calculated for each iFU application (Mourad 1999).

Complete Freund's adjuvant (CFA) was injected into one rear paw of test animals (rats) to produce inflammation. Beginning five days after CFA injection, the animals were habituated to the iFU transducer and test environment. One week following CFA injection, iFU pulses of varying intensity and duration were applied in increasing doses to both the inflamed and the untreated paws until a paw withdrawal threshold was induced. Ultrasound gel was applied to the iFU probe as frequently as necessary to ensure adequate acoustic coupling. No animals were tested more frequently than every 30 seconds and initial withdrawal responses were verified by administration of the same protocol a second time following the rest period to confirm withdrawal. In the absence of a confirmed withdrawal response, the acoustic power of the iFU pulse was increased from 1.5 W to 3 W to 6 W to 9 W to 13.5 W to 20 W to 27 W to 35 W to 42 W to 49 W until a confirmed withdrawal response was observed. iFU withdrawal thresholds on both inflamed and untreated paws were assessed as pulse durations of 0.1, 0.2, 0.33 and 0.5 seconds.

A subset of animals was tested for hind paw withdrawal to heat using a modified Hargreaves test (Hargreaves et al., 1988). A test animal group was also subjected to both HIFU and Hargreaves withdrawal tests. Another subset of test animals was tested for a withdrawal response using the modified Hargreaves test and with a sham (no power) iFU test.

Some untreated animals didn't exhibit an iFU withdrawal response for either paw, even at the maximum acoustic power achievable by the experimental device. In general, longer duration pulses produced more withdrawal responses. The majority of test (CFA injected) animals demonstrated an iFU withdrawal threshold regardless of the pulse duration, and a majority of those animals withdrew the damaged paw at the threshold dose. The administration of the Hargreaves test did not alter the iFU threshold dose when averaged over all acoustic protocols, or within a given acoustic protocol. Acoustic dose was calculated as the acoustic intensity multiplied by the total time the iFU was activated.

In test (CFA injected) animals, the iFU withdrawal threshold in the inflamed paw was significantly lower than that that of the (contralateral) untreated paw. These data correlated with paw withdrawal thresholds to heat measured using the Hargreave's test. Repeated Hargreave's testing produced no change in iFU thresholds, and the results were repeatable from day to day. Repeated iFU testing at threshold levels produced no change in either iFU or Hargreave's test thresholds, and iFU treatments did not produce any evident long-term changes in sensory or motor behaviors of the test animals.

Using the results from the iFU withdrawal tests, the sensitivity and specificity of this procedure was calculated. The sensitivity was defined as the true positive value or the probability that a screening test is positive given that the person has the disease. This was calculated by dividing the number of true positive responses by the sum of the true positive and the false negative responses. The specificity was defined as the true negative rate or the probability that a screening test was negative given that the person does not have the disease. Specificity was calculated by dividing the number of true negative responses by the sum of the true negative and false positive responses.

Calculated values for sensitivity and specificity for the iFU withdrawal test in those animals for which an iFU withdrawal threshold dose could be defined were quite high, generally well over 90%. The longest iFU pulse duration demonstrated reduced values for sensitivity and specificity relative to other iFU protocols, but the sensitivity and specificity were still over 90%. Thus, the iFU withdrawal test demonstrated validity in differentiating normal from abnormal tissue with good sensitivity and specificity. It was reliable, as demonstrated with repeated testing and it does not produce any evident tissue damage.

Tissue damage was assessed by examining the behavior and morphology of hind paws following iFU administration, as well as a lack of interaction with the more common plantar hind paw radiant heat withdrawal (“Hargreaves” test). Hargreaves testing did not influence iFU thresholds and was not affected by iFU testing. In particular, animals receiving both Hargreaves and iFU generally showed statistically the same threshold doses of iFU as the animals that received iFU only.

### Example 3

An experimental protocol was developed to demonstrate that intense focused ultrasound (iFU) can detect peripheral neuropathic pain in the extremity of an animal model of pain. Partial sciatic nerve ligations (pSNL, protocol described in Seltzer et al., 1990 Z. Seltzer, R. Dubner and Y. Shir, A novel behavioral model of neuropathic pain disorders produced in rats by partial sciatic nerve injury, Pain 43 (1990), pp. 205-218) were performed on one group of Sprague Dawley rats on one of their two hind paws, thereby sensitizing that paw.

The prototype iFU device consisted of a commercial piezo-electric, flat transducer built into a solid, cylindrical cone shaped aluminum housing whose dimensions allowed the ultrasound emitted from the transducer to have a focus at the proximal tip of the aluminum housing. The focus of the device was characterized with a needle hydrophone to measure the spatial peak and temporal peak intensity (I_sptp), as described in Miao et al. (2005). The focus of the iFU device was about the size of a grain of rice, extending out less than a centimeter from the transducer head and with a width of less than half a centimeter.

The solid cone device was driven by two function generators (33120A, Hewlett Packard/Agilent, Palo Alto, Calif.) and an amplifier (A150 RF Power Amplifier, ENI, Rome, Italy). The first function generator's role was to gate the pulse to a specific duration. The second function generator, in series with the first, was used to modify the acoustic output and ensure that the pulse was emitted at a specific frequency. The amplifier increased the signal from the function generators and sent it to the solid cone device. An oscilloscope (Wave Runner LT 322, LeCroy, Chesnut Ridge, N.Y.) measured the duration of the pulse, its carrier frequency and the voltage delivered to the iFU device by the amplifier. This voltage was correlated to acoustic intensity emitted by the iFU device via a ‘force balance’ technique, whereby the displacement of a scale produced by ultrasound energy emitted by the device, along with geometric measurements of the spatial distribution of ultrasound energy, were translated mathematically into the spatial and temporal average of acoustic intensity (I_sata).

Approximately one week following the pSNL, iFU was applied in increasing doses to each rear paw until the animal consistently withdrew either paw from the iFU applicator, thereby identifying the iFU threshold dose for that animal. Additional iFU applications were then performed at the threshold dose for each given rat to determine the sensitivity and specificity of the iFU application at the threshold dose. In addition, each animal's response to the Hargreaves (heat lamp) test, applied both before and after the iFU threshold dose test, was observed as a functional assay of the safety of iFU administration.

Individual pulses of ultrasound were used in these experiments. The center frequency was 1.15 Mhz, with pulse duration of 0.2 seconds. On each day of experiments, we calibrated the iFU device using the force balance noted above. Using the value of I_sata derived in that fashion, we calculated the acoustic dose (I_sata multiplied by ultrasound pulse duration) for each IFU application. After completing the habituation of a set of three rats, each housed in an individual cage, we measured the iFU withdrawal thresholds of each hindpaw of each of the rats. Starting at 1.5 watts (W) of acoustic power, we applied ultrasound to one plantar hindpaw of each rat in series. The focus of the iFU stimulates both superficial and deep tissue of the rat's paw. Immediately after iFU application we observed the animals, looking for any withdrawal responses following the iFU pulse, before returning to the other hindpaw in each of the three rats. Two independent observers agreed on all withdrawal response or non-response behaviors; in there was not agreement, the trial was repeated. No rats were tested more frequently than every 30 seconds and ultrasound gel was placed on the transducer tip as often as necessary to ensure adequate physical coupling of the device to the plantar aspect of the rats' paws.

In the absence of a withdrawal response, the acoustic power was increased from 1.5 W through 3 W, 6 W, 9 W, 13.5 W, 20 W, 27 W, 35 W, 42 W to 49 W or until a withdrawal response was observed. If a withdrawal of one paw of a given rat was observed, iFU was applied again to that paw after waiting 30 seconds, with the same acoustic power. If the rat withdrew its paw again, the associated acoustic parameters were recorded (I_sata and the duration of iFU application, in seconds) as the “iFU threshold dose” for that paw. Each rat hindpaw was again tested with that rat's iFU threshold dose an additional 6 times to confirm consistent paw withdrawal and to verify the iFU threshold dose for each animal.

Rats showing at most only one out of two withdrawal responses to a given level of iFU stimulation at a given power were considered negative tests and the power was increased until the iFU power at a given duration induced two consecutive withdrawal responses from a given paw. Rarely were rats observed to withdraw each of its hindpaws to a given iFU dose. In these rare cases, the acoustic power was decreased rather than increased, and the iFU protocol resumed until the paw withdrawal was observed twice, as described above. If, during this threshold measurement procedure, a rat began to withdraw its paw in response to contact with the transducer but without iFU application, the rat was re-habituated to the touch of the device before resuming iFU application. The iFU threshold test was performed on both hindpaws on each rat.

FIG. 8 shows the acoustic dose necessary to produce an unambiguous withdrawal response by all pSNL rats (both Dropped and Non-dropped paw pSNL rats), as well as control rats (both Sham and Naïve (“Control”)) after application of iFU to either of their sensitized or normal paws. ‘All pSNL’ refers to all rats who had the full ligation surgery; ‘Dropped’ refers to those rats who showed all of the symptoms necessary to demonstrate a successful surgery; ‘Non-dropped’ refers to those rats who did not show all of the symptoms necesssary to demonstrate a successful surgery; ‘Sham’ refers to those rats who underwent sufficient surgery to expose and stretch the sciatic nerve, without ligation of the nerve; ‘Control’ refers to those (naïve) rats that did not receive any surgical procedure. Once an animal underwent even sham surgery (let alone full ligation), both of their paws become sensitive to iFU stimulation such that iFU could differentiate between the sensitized and control paw of the ‘Dropped’ animals with sensitivity and specificity around 85%. The threshold acoustic doses required to induce a paw withdrawal response were significantly different from (and lower than) the acoustic doses observed in the Control (naïve) animals.

The results demonstrated that 58 of the 59 “Dropped paw” rats subjected to the full pSNL surgery consistently withdrew their injured rather than their control paw with application of sufficient iFU, with sensitivity and specificity of approximately 85%. iFU threshold doses were consistent across applications and both before and after application of the Hargreaves test; Hargreaves latency times did not change after iFU application. iFU application thus provided discrimination of peripheral neuropathic versus control tissue. The stability of the Hargreaves test results over time suggests that the iFU threshold test is a safe method of detecting and identifying peripheral neuropathic tissue.

### Example 4

Several human subjects were probed with iFU acoustic radiation forces using an experimental iFU transducer similar to that described above to assess individual sensitivity levels to acoustic doses and to determine appropriate acoustic doses for evaluating a human subject's sensitivity and localizing pain. Acoustic doses were calculated as the acoustic intensity multiplied by the total time the iFU was activated. Twenty sham or actual iFU applications were performed for each acoustic dose for each of the volunteer's two index fingers so that HIFU application was effectively blinded. After each application, subjects were asked if they felt anything. If the answer was no, the test continued. If the answer was yes, the volunteers were asked to describe the sensation and if it was uncomfortable or painful, they were asked to rate their pain on a subjective scale of 1-10, with 10 being the most painful sensation they'd ever experienced. After each set of twenty applications, the intensity of the iFU was increased and another round of palpations was commenced. Varying acoustic doses were applied for a duration of 0.1 second until a subjective sensitivity was reached.

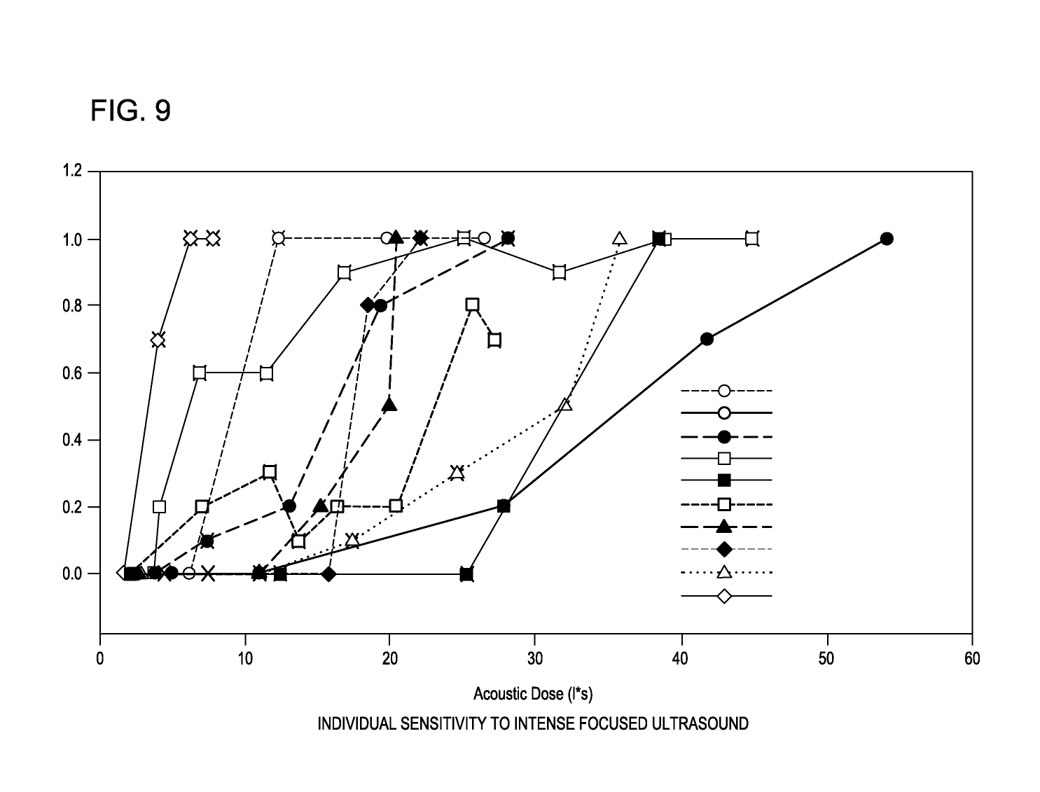

The data collected for the first volunteer indicated that reliable sensitivity and specificity required an iFU acoustic dose of at least 15 (W/cm²)*sec. The iFU threshold for acoustic palpation sensation in this volunteer was approximately 16 (W/cm²)*sec—determined as the acoustic dose necessary to reliably (and with a sensitivity and specificity greater than 90%) generate a sensation in this volunteer. Another three volunteers who underwent the same test had an iFU threshold dose for acoustic palpation sensation, as defined above, of 12, 26 and 55 (W/cm²)*sec. Another volunteer, who underwent a comparable procedure on a single index finger, had an iFU threshold for acoustic palpation sensation of 20 (W/cm²)*sec. The results for sensitivity are shown graphically in FIG. 9. The specificity was always at least 80%.

These thresholds for human sensation are of the same order as the acoustic dose necessary to generate a withdrawal response in the inflamed paws of the rat. All volunteers described the sensation as a mechanical one—a “push” or a “thump.” For some, the sensation was sharp, like a needle, though not as painful. For others the sensation was blunt, like the push of an eraser. One volunteer also reported an itchy sensation, and another described “cold heat.” Two reported a dual sensation—a “push” followed by an increasing, warm sensation that was never hot.

### Example 5

Subsequent experiments were performed by applying iFU at a frequency of 1.1 MHz for 0.1 sec to the fingertip pads of seventeen human test subjects in a blinded fashion and escalating intensities until the subjects consistently observed iFU-induced sensations. Most of the test subjects achieved high values of sensitivity and specificity at values of spatially and temporally averaged intensity measuring less than 100 W/cm². We also concluded that the test subjects' sensitivity to iFU stimulation correlated with the density of mechanoreceptors as determined by a two-point neurological examination.

The specifics of the device used for these experiments are described in detail in Miao et al. (2005). The prototype device consisted of a commercial flat, piezo-electric transducer built into a custom made, solid, cylindrical cone shaped aluminum housing. The focus of the ‘solid cone’ device occurred near the surface of the device that was placed on the finger pads of the volunteers, with secondary maxima at 0.75 and 1.75 mm. These foci were characterized with a needle hydrophone in water. The width of the primary focus as measured at the half-pressure-maximum contour measured 1.7 mm and was also characterized via needle hydrophone. The solid cone device was driven by two function generators (33120A, Hewlett Packard/Agilent, Palo Alto, Calif.) and an amplifier (A150 RF Power Amplifier, ENI, Chesnut Ridge, N.Y.). An oscilloscope (Wave Runner LT 322, LeCroy, Chesnut Ridge, N.Y.) measured the duration of the pulse, its carrier frequency and the voltage delivered to the iFU device by the amplifier during each experiment. This voltage was correlated to acoustic intensity emitted by the iFU device via a ‘force balance’ technique described by Hill et al. 1994, Sutton et al. 2006. Using the force balance displacement along with the geometric specifics of our device we calculated a measure of intensity (I_{SAL}). Specifically, I_{SAL} is the spatially averaged and temporally averaged intensity over the area enclosed by the half-pressure-maximum contour in the focal plane, a standard measure of ultrasound intensity described and justified by Hill et al. (1994).

The fundamental frequency for the transducer was 1.117 MHz. For all experiments, we used a single pulse of ultrasound with a duration of 100 milliseconds. The study group consisted of 17 test subjects (10 male, 7 female), ages 18-56. The iFU and the “two-point” neurological testing were conducted on two separate days, at least one week apart as described below. All subjects were screened for any history that would associate them with abnormal sensory phenomena.

The two-point discrimination test, a standard neurological assay for finger sensitivity, was performed on the index finger of each of the subject's hands, using the DISK-CRIMINATOR™ (Dellon, Baltimore, Md.). The test depends on the density of mechanoreceptors within the peripheral nerves that enervate the finger-tip pads (Johansson and Vallbo 1979, Mackinnon and Dellon 1985). The subjects were also given a questionnaire related to any recent pain in the hand or abnormal sensations that they had experienced. The questionnaire and two-point test were given both before and after iFU testing and helped to determine whether any short-term physiological or psychological perception changes were associated with the iFU stimulation tests.

After completing the questionnaire and two-point test the subject was seated and familiarized with the ultrasound device as well as the testing equipment and associated test protocol. Next, the test subjects were asked to place the pad of either the right or left index finger on the cone tip. Ultrasound gel (Aquasonic 100 Ultrasound Transmission gel, Parker Laboratories, Fairfield, N.J.) was used to ensure adequate acoustic transmission. The tester would alert the subjects that they were about to receive a potential iFU pulse. The participants were blinded, however, as to whether or not the iFU stimulus was actually delivered or a sham application was performed instead. The test subjects were then immediately asked if they felt a sensation. If a subject reported pain they were asked to rate it on the 11-point numeric scale (NRS 0-10). A computer program randomized each trial of 20 pulses for which finger was used (10 on left and 10 on right) and whether a sham pulse or a real pulse was given (10 real and 10 sham pulses per iFU intensity value).

We used the method of ascending limits to determine the amount of ultrasound necessary to generate a sensation with a 90% sensitivity value (Snodgrass, 1975). We call this amount the ‘90% threshold intensity of iFU’. We began testing at a sub-perceptive threshold and systematically increased the iFU intensity every trial of 20 applications until either: the subject reported 9 out of 10 true positives, the ultrasound output reached 650 W/cm² (a value determined during separate animal studies to produce reliable and safe sensations in the paws of sensitized rats) or we ran out of time for a given subject. A second test session was performed a minimum of one week after the first. This second test followed the same procedures as the first. This second test was used to identify possible long-term effects on perception as well as the consistency of response by the subjects to the ultrasound stimulus.

In all cases the data was analyzed using student's t-test for significance, with a p value of less than 0.05. As well, we used the metrics of sensitivity and specificity to analyze the data, where sensitivity refers to the number of true positives registered by the subject, defined as one minus the rate of Type I ‘False Positive’ errors, and specificity refers to the number of true negatives registered by the subject, defined as one minus the rate of Type II ‘False Negative’ errors (Snodgrass, 1975).

We observed no significant difference between the average two-point threshold values before and after iFU testing nor between the first and second sessions. As well, the pre- and post-test questionnaire showed no psychological or physiological changes associated with the testing process. Out of 34 tests, 5 tests reported transient pain, with the median value reported as NRS 1/10. The median number of times a subject reported pain was also only once per testing period. None of the subjects complained about any long-lasting effects. As well, we did not observe any statistically significant differences for the 90% threshold intensity value of iFU between the first and the second sessions; we therefore combined the two sessions' worth of results for subsequent analysis.

Our group of 17 subjects and 34 tests achieved an average two-point value of 2.83 mm+/−0.79. Due to time constraints, 25 of 34 tests were able to reach the 90% threshold intensity value of iFU. The average intensity for the attainment of the 90% threshold value for those members who did attain a threshold was 106.4+/−58.7 W/cm². All subjects reached a sensitivity value of 50% within the time provided. The average value of iFU intensity at 50% sensitivity was 95+/−69.7 W/cm². The results demonstrated a sigmoidal distribution of iFU sensation threshold versus iFU intensity, which is consistent with other stimuli such as mechanical vibration and thermal stimulation. The specificity of subjects' responses to iFU testing remained high throughout the entire process, with an average value of 94% (a 6% false positive rate). The vast majority of sensations (>99%) were pain free, with the median pain score measuring one out of 10.

### Example 6

Experimental studies were conducted in an animal model to determine whether iFU could be used to induce temporal summation in damaged tissue. Ultrasound was generated using the inner element (22.6 mm inner diameter, 48.5 mm outer diameter) of a two-element, 2 MHz annular array transducer (H-106 S/N-01, Sonic Concepts, Inc., Woodinville, Wash.), placed within a brass housing that facilitated hand-held deployment of the device. The geometric focus of this device is 62.6 mm away from the transducer. The dimensions of the focus were approximately 1.2 mm in diameter and 1 cm in length (at the half pressure maximum contour) along the axis of acoustic propagation. A plastic cone filled with degassed water was used to transmit the ultrasound to the focus for the animal studies. The base of the cone was sealed to the transducer housing, and the truncated apex of the cone was sealed with a thin sheet of stretched latex. The height of the cone put the center of the ultrasound focus 6.9 mm beyond the proximal surface of our device, as measured in degassed water. The ultrasound transducer was driven by two arbitrary waveform generators (33120A, Agilent Technologies Inc., Palo Alto, Calif.) and an A150 power amplifier (ENI, Rochester, N.Y.). The first waveform generator controlled the pulse duration and pulse repetition frequency by gating the 2 MHz sinusoidal output of the second waveform generator. The output of the gated waveform generator was amplified by the power amplifier, which was connected to the transducer via an impedance matching network.

Prior to any studies, the acoustic output of the transducer as a function of input voltage was measured using a radiation force balance (Poliachik et al. 2001). During studies, input voltage to the transducer was recorded using a Wave Runner LT 322 oscilloscope (LeCroy Corporation, Chestnut Ridge, N.Y.). The acoustic beam pattern of the transducer was numerically computed in MATLAB (The Mathworks, Natick, Mass.) assuming linear wave propagation. Using the data from the beam pattern, the input voltage to the transducer and the total acoustic output measured by the radiation force balance, we calculated the intensity at the focus. Results were reported in terms of I_{SAL}, the spatially averaged, temporally averaged intensity over the area enclosed by the half-pressure-maximum contour in the focal plane assuming linear conditions, a standard measure of ultrasound intensity described and justified by Hill et al (1994). We also measured the acoustic output in terms of the acoustic dose—the product of I_{SAL} and the total iFU temporal duration, which, for this study, always measured 0.1 seconds.

Inflammation was induced in one rear paw of test animals, the animals were habituated to the iFU, the ultrasound probe and their surroundings, and each animal underwent a set of Hargreaves tests to assay the sensitivity of the test animals to rapid application of cutaneous heat. Starting at an acoustic intensity (I_{SAL}—spatially averaged, temporally averaged intensity, defined below) of approximately 100 W/cm² with a duration of 0.1 seconds ultrasound was applied to the plantar surface of one hind paw, chosen randomly, of each rat in series. During and immediately after iFU application we observed the animals, looking for any immediate hind paw withdrawal response, before returning to the other hind paw in each of the rats. We then repeated that study for the other paw of the same rats. No rats were tested more frequently than every 30 seconds and ultrasound gel was placed on the transducer tip as often as necessary to ensure adequate physical coupling of the device to the plantar surface of the rats' paws.

In the absence of an observed hind paw withdrawal response we increased the intensity of ultrasound and performed the experiment again. We did so in increments of approximately 10-30%, with smaller increments as the intensity increased, until we observed a withdrawal response. However, we never increased the acoustic intensity above 1500 W/cm² because we observed the ultrasound gel to cavitate on top of the device at this intensity.

If we observed a withdrawal of one paw of a given rat to a given amount of ultrasound, we applied that iFU protocol again to that paw after waiting 30 seconds. If the rat withdrew its paw again, we recorded the associated acoustic parameters—I_{SAL} and the duration of the iFU application, in seconds—as the iFU threshold dose and iFU threshold intensity for that paw—taken together as the iFU threshold value. Then each of that rat's hind paws was again tested with that rat's iFU threshold value for an additional 6 times looking for paw withdrawal. We used that data to calculate the sensitivity and specificity of the ability of iFU to induce a withdrawal response. We refer to this complete test of determining an iFU threshold value followed by the additional 6 iFU withdrawal tests as the “iFU test”.

Rats showing only one out of two withdrawal responses to iFU stimulation at a given intensity were considered negative tests and the intensity was increased as above, until we identified the minimum iFU value that induced two consecutive withdrawal responses from a given paw. Rarely were rats observed to withdraw both of its hind paws to a given iFU value. In these rare cases, we decreased, rather than increased, the iFU value, and re-applied iFU as above until we observed only one paw withdraw twice.

The iFU test was performed on both hind paws on each rat for the acoustic protocols (testing one protocol per rat per day) on days 5, 7, 12, and 14 post-CFA injection. We performed this sequence of tests on 19 rats, obtaining a total usable data set containing 49 points. Each rat was tested with at least 2 out of the 3 ultrasound protocols described below on rotating days.

Three different acoustic protocols were used. The first acoustic protocol consisted of a single pulse measuring 75 ms in duration (the ‘brief acoustic protocol’ or BAP. The second acoustic protocol consisted of a train of five such pulses, each measuring 75 ms separated by 75 ms during which the ultrasound is off (the ‘temporal summation acoustic protocol’ or TSAP). This puts the frequency of the iFU repetition at 6.66 Hz. The third acoustic protocol consisted of a single long pulse equal in duration to the time that the ultrasound is on the TSAP protocol (the ‘single, long acoustic protocol’ or SLAP).

The TSAP (the temporal summation acoustic protocol), SLAP (the single, long acoustic protocol) and BAP (the brief acoustic protocol) protocols were first applied to normal rat's paws. Thresholds for TSAP and SLAP were determined, but BAP thresholds could not be determined at an intensity lower than 1500 W/cm², the maximum intensity used. The results demonstrated that the TSAP threshold for normal tissue is statistically significantly higher than SLAP, while BAP is significantly higher than both. In terms of acoustic dose, the results showed that SLAP is significantly lower than TSAP.

The same studies were then conducted on animals with a paw inflamed via CFA injection. The CFA animals had thresholds significantly lower than the normal animals, and the data followed similar trends as were seen in the normal animals. That is, in terms of intensity, BAP was significantly higher than both TSAP and SLAP, and TSAP was significantly higher than SLAP. In terms of acoustic dose, TSAP was significantly higher than SLAP, while BAP was no longer statistically significantly different from TAP. The sensitivity and specificity of the results was calculated: the average value across all acoustic protocols for CFA animals was 88+/−12% for sensitivity and 87+/−19% for specificity and did not vary in a significant way as between the acoustic protocols. The average Hargreaves latency time for all protocols was 10.1+/−5 seconds for the abnormal paw and 17.7+/−3 seconds for the normal paw. The latency times did not vary in a significant way as between the acoustic protocols, nor did they vary before versus after the iFU test.

These studies suggested that iFU thresholds for various protocols can be reliably identified and that the iFU protocols can be safely used to induce temporal summation in damaged or inflamed tissue.

### Example 7

A laser guided acoustic palpation device was constructed for probing internal tissue sites. The laser-guided device consisted a 2 MHz single-element focused transducer (Sonic Concepts, Bothell, Wash.) on which a removable plastic cone was mounted. The cone was filled with degassed water and then capped with a latex membrane for transmitting the ultrasound from the transducer into the tissue. Two (2) lasers (Digi-Key, Thief River Falls, Minn.) were mounted permanently on the sides of this cone, pointed at the center of the ultrasound focus. The transducer was driven by two function generators (33120A, Hewlett Packard/Agilent, Palo Alto, Calif.) and an amplifier (A150 RF Power Amplifier, ENI, Chesnut Ridge, N.Y.). The first generator gated the pulse to a specific duration. The second, in series with the first, modified the acoustic output and ensured that the pulse was emitted at a specific frequency. The amplifier increased the signal from the function generators and sent it to the cone device. An oscilloscope (Wave Runner LT 322, LeCroy, Chesnut Ridge, N.Y.) measured the duration of the pulse, its carrier frequency and the voltage delivered to the iFU device by the amplifier during each experiment.

Data was collected using the laser-guided system described above, as well as a shallow-focused acoustic palpation device and a more deeply focused acoustic palpation device using the animal model described previously to define the sensitivity and specificity of iFU application. Experimental data showed that the threshold intensity and dose values were significantly higher for the animals with normal paws versus the animals with inflamed paws. The data also showed that the threshold intensity and dose values were significantly higher for the deep versus shallow iFU stimulation protocols. This experimental work supports the use of iFU stimulation protocols to differentiate between injured and uninjured tissue at deep tissue sites, and suggests that painful responses (and chronic pain syndromes) associated with deep tissue sites may be quantifiable using iFU stimulation protocols.

We claim:

1. A method for assessing a subject with chronic pain, comprising: selecting at least one localized target site internal to the subject's external body surface and within an area of sensitized tissue; applying intense focused ultrasound pulses having different acoustic properties to the localized target site; and assessing the duration and/or intensity and/or quality of the subject's responses to the focused ultrasound pulses.

2. The method of claim 1, additionally comprising applying intense focused ultrasound pulses to a plurality of localized target sites within the area of sensitized tissue.

3. The method of claim 2, additionally comprising mapping pain responses elicited by application of intense focused ultrasound to the plurality of localized target sites.

4. The method of claim 1, wherein the different acoustic properties are selected from the group consisting of: magnitude, frequency, intensity, duration and pulse repetition rate.

5. The method of claim 1, additionally comprising comparing the duration and/or intensity and/or quality of the subject's responses to a predetermined standard response profile.

6. The method of claim 1, additionally comprising performing the application of intense focused ultrasound pulses to the at least one localized target site prior to and subsequent to the administration of a treatment regimen to assess the success or failure of the treatment regimen.

7. The method of claim 1, additionally comprising performing the application of intense focused ultrasound pulses to the at least one localized target site periodically to assess the progression or amelioration of chronic pain.

8. A method of claim 1, comprising applying intense focused ultrasound pulses to localized target sites using multiple ultrasound sources by converging multiple acoustic beams at a localized target site.

9. A method of claim 1, additionally comprising providing an instrument to the subject that allows the subject to indicate sensations perceived in response to intense focused ultrasound pulses and recording the sensations perceived by the subject during probing.

10. A method of claim 1, additionally comprising detecting at least one of the following physiological parameters prior to, during and/or following application of the intense focused ultrasound pulses: respiration, heart rate, overall body temperature, tissue temperature at the target site, electrical heart activity, blood flow velocity, blood pressure, intracranial pressure (ICP), blood flow-related irregularities, electrical brain activity, skin conductance or impedance, and blood oxygen composition or partial pressure (pO₂, pCO₂).

11. A method of claim 1, additionally comprising selecting from among a plurality of predetermined routines for applying intense focused ultrasound pulses having different acoustic doses to a localized target site.

12. A method for localizing nerves and other sensitized tissues for guidance of delivery devices and/or compositions to nerve sites and sensitized sites comprising: locating a generalized target area incorporating the anticipated site of a nerve or sensitized tissue; applying intense focused ultrasound pulses to a plurality of localized target sites within the generalized target area; and spatially identifying a localized target site that produces a detectable sensation or biological response indicating the presence of a nerve or sensitized tissue.

13. The method of claim 12, additionally comprising guiding a delivery device to the localized target site that produces a detectable sensation or biological response indicating the presence of a nerve or sensitized tissue.

14. The method of claim 13, wherein the delivery device is a needle.

15. The method of claim 12, additionally comprising delivering a composition to the localized target site that produces a detectable sensation or biological response indicating the presence of a nerve or sensitized tissue.

16. The method of claim 15, wherein the composition is selected from the group consisting of an anesthetic composition, an analgesic composition, and a treatment composition.

17. A method for assessing a physiological parameter of a target tissue, comprising the steps of:
(a) applying a pulse of focused ultrasound to a first target tissue site thereby inducing oscillation of said first target tissue site;
(b) measuring a property of an acoustic signal emitted from said oscillating first target tissue site; and
(c) relating the property of the emitted acoustic signal to a physiological tissue property.

18. The method of claim 17, wherein said target tissue is peripheral nervous system tissue.

19. The method of claim 17, further comprising the steps of:
(a) applying focused ultrasound to a second target tissue site thereby inducing oscillation of said target tissue site;
(b) measuring a property of an acoustic signal emitted from said oscillating second target tissue site; and
(c) comparing said property of an acoustic signal emitted from said oscillating second target tissue site to said property of an acoustic signal emitted from said oscillating first target tissue site; and
(d) relating said compared properties to a physiological tissue property.

20. The method of claim 19, wherein said applied focused ultrasound to said first target tissue site and to said second target tissue site comprises a plurality of acoustic interrogation pulses to said first and said second target tissue sites.
